(19) United States
(12) Patent Application Publication
(10) Pub. No.: US 2018/0036364 A1
(43) Pub. Date: Feb. 8, 2018

(54) METHODS OF TREATING CANCER WITH A PSMA LIGAND-TUBULYSIN COMPOUND

(71) Applicant: ENDOCYTE, INC., West Lafayette, IN (US)

(72) Inventors: Christopher Paul LEAMON, West Lafayette, IN (US); Binh NGUYEN, Indianapolis, IN (US)

(21) Appl. No.: 15/554,866

(22) PCT Filed: Mar. 1, 2016
(86) PCT No.: PCT/US16/20238

§ 371 Date: Aug. 31, 2017

Related U.S. Application Data
(60) Provisional application No. 62/289,298, filed on Jan. 31, 2016.
(60) Provisional application No. 62/126,635, filed on Mar. 1, 2015.

(51) Int. Cl. A61K 38/06; A61K 51/08

(52) U.S. Cl. CPC A61K 38/06; A61K 51/088

(57) ABSTRACT

The invention described herein pertains to drug delivery conjugates for targeted therapy. The invention described herein relates to methods of treating PSMA expressing cancers with a PSMA ligand-tubulysin compound. The invention described herein also relates to methods of treating PSMA-expressing cancers with a PSMA ligand-tubulysin compound in patients where stable disease results after treatment with the PSMA ligand-tubulysin compound.

### CROSS REFERENCE TO RELATED APPLICATIONS

This application claims priority under 35 U.S.C. §119(e) to U.S. Provisional Application Ser. No. 62/126,635, filed Mar. 1, 2015 and U.S. Provisional Application Ser. No. 62/289,298, filed Jan. 31, 2016, in which all of which are incorporated herein by reference in their entirety.

### FIELD OF THE INVENTION

The invention described herein pertains to drug delivery conjugates for targeted therapy. The invention described herein relates to methods of treating PSMA expressing cancers with a PSMA ligand-tubulysin compound. The invention described herein also relates to methods of treating PSMA-expressing cancers with a PSMA ligand-tubulysin compound in patients where stable disease results after treatment with the PSMA ligand-tubulysin compound.

### BACKGROUND

Prostate specific membrane antigen (PSMA) is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. PSMA plays a role in the central nervous system, where it metabolizes N-acetyl-aspartyl glutamate (NAAG) into glutamic and N-acetyl aspartic acid. Accordingly, it is also sometimes referred to as an N-acetyl alpha linked acidic dipeptidase (NAALADase). PSMA is also sometimes referred to as a folate hydrolase I (FOLH I) or glutamate carboxypeptidase (GCP II) due to its role in the proximal small intestine where it removes γ-linked glutamate from poly-γ-glutamated folate and α-linked glutamate from peptides and small molecules.

PSMA is named largely due to its higher level of expression on prostate cancer cells; however, its particular function on prostate cancer cells remains unresolved. PSMA is over-expressed in the malignant prostate tissues when compared to other organs in the human body such as kidney, proximal small intestine, and salivary glands. Unlike many other membrane-bound proteins, PSMA undergoes rapid internalization into the cell in a similar fashion to cell surface bound receptors like vitamin receptors. PSMA is internalized through clathrin-coated pits and subsequently can either recycle to the cell surface or go to lysosomes. It has been suggested that the dimer and monomer form of PSMA are inter-convertible, though direct evidence of the interconversion is being debated. Even so, only the dimer of PSMA possesses enzymatic activity, and the monomer does not.

Though the activity of the PSMA on the cell surface of the prostate cells remains under investigation, it has been recognized by the inventors herein that PSMA represents a viable target for the selective and/or specific delivery of biologically active agents, including drug compounds and imaging agents to such prostate cells. One such drug compound is tubulysin, which when conjugated to PSMA through an appropriately functionalized linker provides PSMA ligand-tubulysin compound I

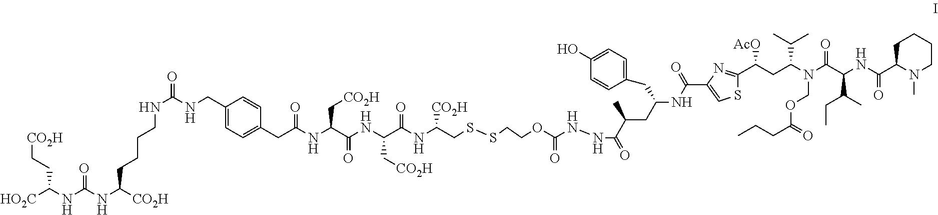 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, useful for the treatment of cancer (also referred to herein as EC1169) as described in WO2014/078484, which is incorporated herein by reference. One such imaging agent is the PSMA ligand-imaging conjugate of formula IIIa

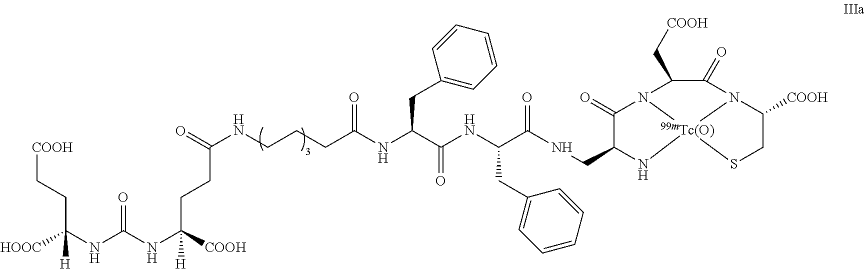

(also referred to herein as ^{99m}Tc-EC0652 or ^{99m}Tc-Conjugate IIa) as described in WO2009/026177, which is incorporated herein by reference. Imaging conjugate IIIa has found use as a cancer imaging agent as described in, for example, WO2009/026177. One of skill in the art will recognize that imaging conjugate IIIa can exist as syn- and anti-isomers in reference to the relative position of the Tc═O double bond.

Throughout this disclosure, various publications, patents and patent applications are referenced. The disclosures of these publications, patents and applications in their entireties are hereby incorporated by reference into this disclosure.

### SUMMARY

In some embodiments, the present disclosure provides a method is provided for treating a cancer in a patient in need of such treatment comprising, administering to the patient a therapeutically effective amount of a PSMA ligand-tubulysin compound I

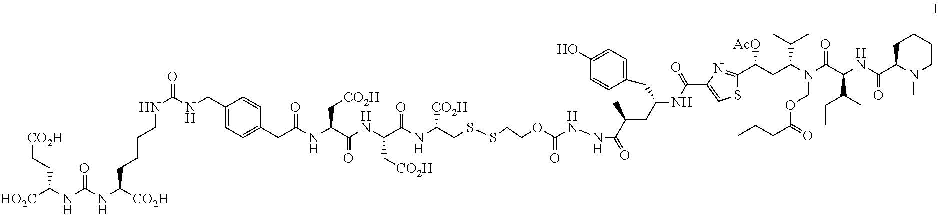 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof.

In some embodiments, the present disclosure provides use of a PSMA ligand-tubulysin compound I

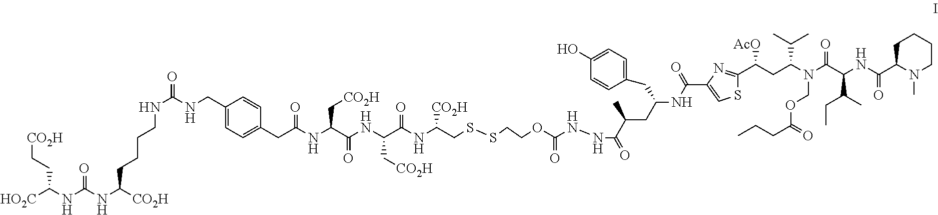 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, for treating a cancer in a patient. In some aspects, the use comprises administering to the patient a therapeutically effective amount of the PSMA ligand-tubulysin compound I, or a pharmaceutically acceptable salt thereof.

In some embodiments, the present disclosure provides use of a PSMA ligand-tubulysin compound I

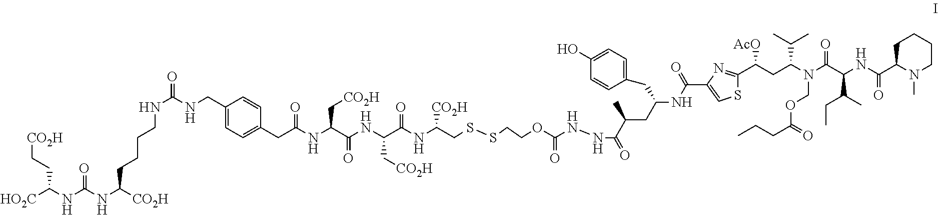 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, in the preparation of a medicament useful for the treatment of a cancer in a patient. In some aspects, the medicament comprises a therapeutically effective amount of the PSMA ligand-tubulysin compound I, or a pharmaceutically acceptable salt thereof.

In some aspects of these embodiments, the cancer is a PSMA expressing cancer. In some aspects of these embodiments, the compound is at least about 98 percent pure. In some embodiments, the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma.

In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction. In some aspects of these embodiments, the cancer is a primary or secondary brain cancer. In some aspects of these embodiments, the cancer is prostate cancer. In some aspects of these embodiments, the cancer is metastatic prostate cancer. In some aspects of these embodiments, the PSMA ligand-tubulysin compound I, or a pharmaceutically acceptable salt thereof, is administered in a parenteral dosage form.

In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 0.1 mg/m² to about 6.0 mg/m². In some aspects of these embodiments, the therapeutically effective amount is from about 0.1 mg/m² to about 5.0 mg/m². In some aspects of these embodiments, the therapeutically effective amount is from about 0.1 mg/m² to about 4.0 mg/m². In some aspects of these embodiments, the therapeutically effective amount is from about 0.1 mg/m² to about 3.5 mg/m². In some aspects of these embodiments, the therapeutically effective amount is from about 0.1 mg/m² to about 3.0 mg/m². In some aspects of these embodiments, the therapeutically effective amount is from about 0.1 mg/m² to about 2.5 mg/m². In some aspects of these embodiments, the therapeutically effective amount is from about 0.1 mg/m² to about 2.0 mg/m².

In some aspects of these embodiments, the therapeutically effective amount is from 0.1 mg/m² to 6.0 mg/m². In some aspects of these embodiments, the therapeutically effective amount is from 0.1 mg/m² to 5.0 mg/m². In some aspects of these embodiments, the therapeutically effective amount is from 0.1 mg/m² to 4.0 mg/m². In some aspects of these embodiments, the therapeutically effective amount is from 0.1 mg/m² to 3.5 mg/m². In some aspects of these embodiments, the therapeutically effective amount is from 0.1 mg/m² to 3.0 mg/m². In some aspects of these embodiments, the therapeutically effective amount is from 0.1 mg/m² to 2.5 mg/m². In some aspects of these embodiments, the therapeutically effective amount is from 0.1 mg/m² to 2.0 mg/m².

In other aspects, the methods and uses described herein further comprise imaging PSMA expression by the cancer. In some aspects of these embodiments, the step of imaging occurs before the step of administering. In some aspects of these embodiments, the imaging is performed by imaging wherein the imaging is selected from the group consisting of SPECT imaging, PET imaging, IHC, and FISH. In some aspects of these embodiments, the imaging is performed by SPECT imaging.

In some aspects of these embodiments, the step of imaging comprises administering to the patient a PSMA ligand-imaging conjugate of the formula II

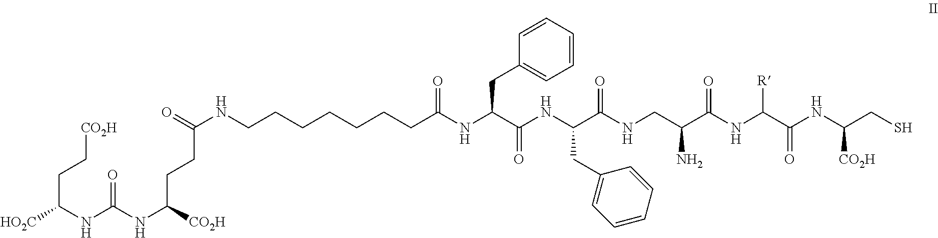

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, the step of imaging comprises administering a PSMA ligand-imaging conjugate of the formula III

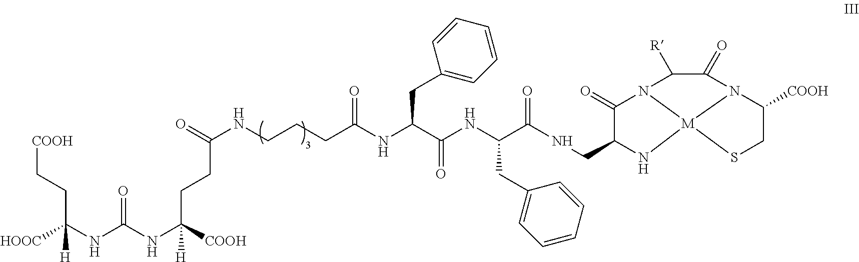

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the PSMA ligand-imaging conjugate is of the formula IIa

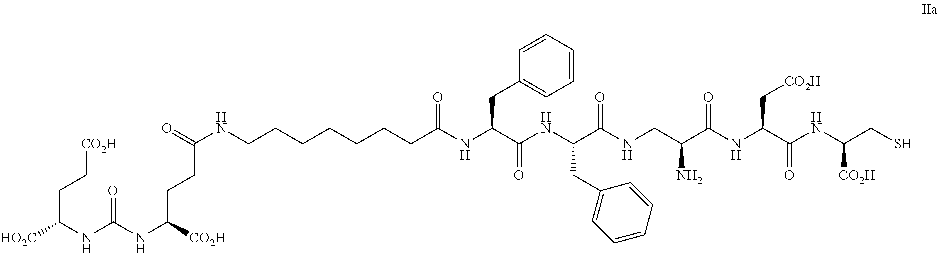

or a pharmaceutically acceptable salt thereof, wherein a radionuclide is bound to the conjugate. In some aspects of these embodiments, the PSMA ligand-imaging conjugate is of the formula IIa

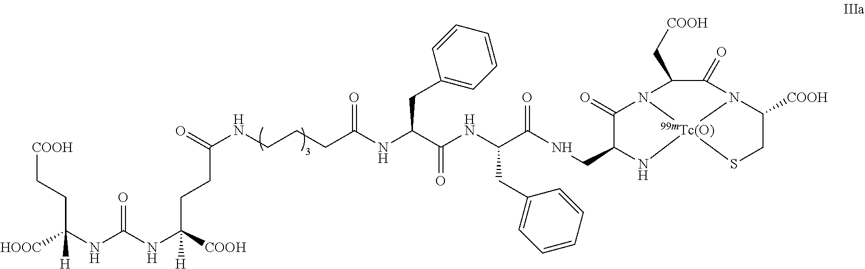

or a pharmaceutically acceptable salt thereof.

In other aspects, the methods and uses described herein further comprise determining the PSMA status of the patient by imaging. In some aspects of these embodiments, the imaging is SPECT imaging. In some aspects of these embodiments, the PSMA status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease. In some aspects of these embodiments, the PSMA positive lesions indicate functionally active PSMA.

In some aspects of these embodiments, the step of determining comprises administering to the patient a PSMA ligand-imaging conjugate of the formula II

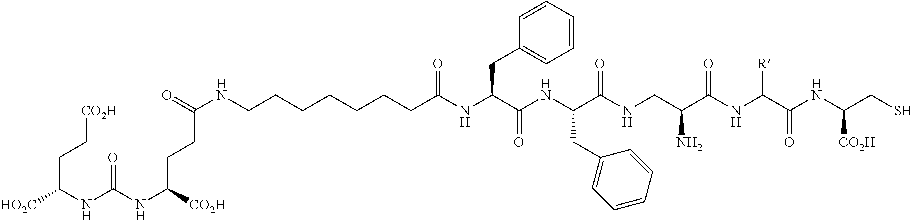

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein the conjugate is bound to a radionuclide.

In some aspects of these embodiments, the step of determining comprises administering a PSMA ligand-imaging conjugate of the formula III

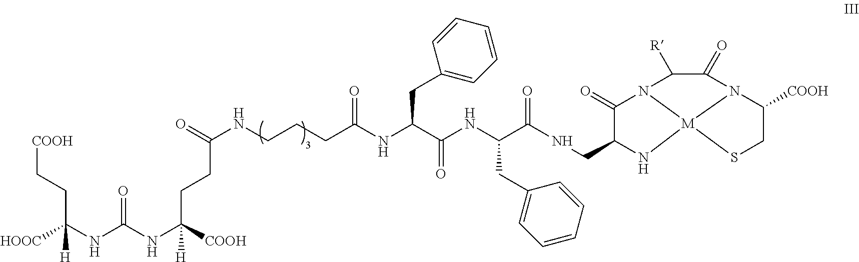

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the PSMA ligand-imaging conjugate is of the formula IIa

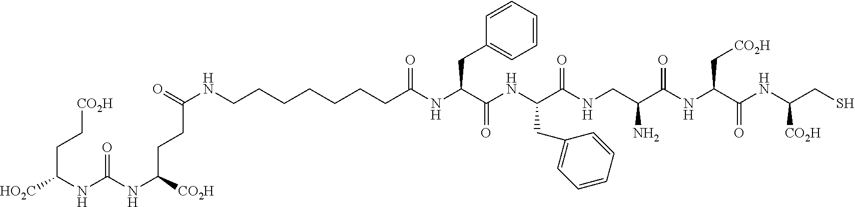 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, wherein a radionuclide is bound to the conjugate.

In some aspects of these embodiments, the PSMA ligand-imaging conjugate is of the formula IIIa

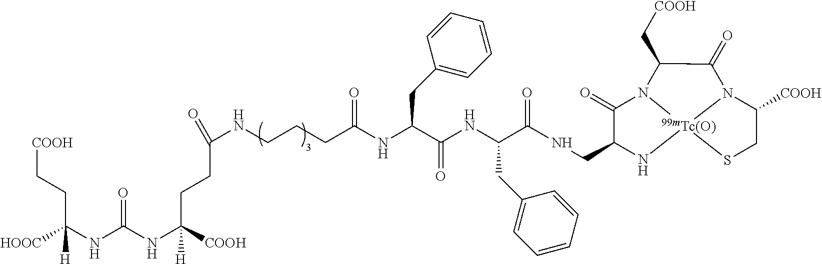

or a pharmaceutically acceptable salt thereof.

In other embodiments, the present disclosure provides a method of treating a cancer in a patient in need of such treatment comprising, administering to the patient a therapeutically effective amount of a PSMA ligand-tubulysin compound I

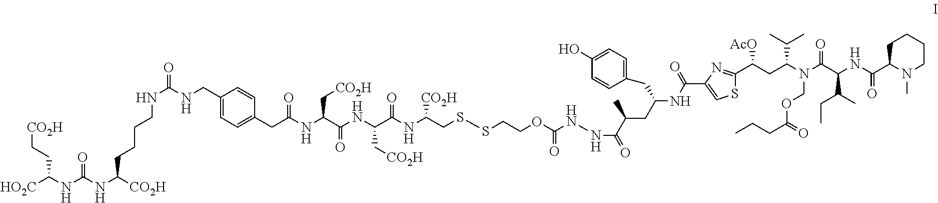 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, wherein stable disease results after the PSMA ligand-tubulysin compound I, or a pharmaceutically acceptable salt thereof, is administered.

In other embodiments, the present disclosure provides use of a PSMA ligand-tubulysin compound I

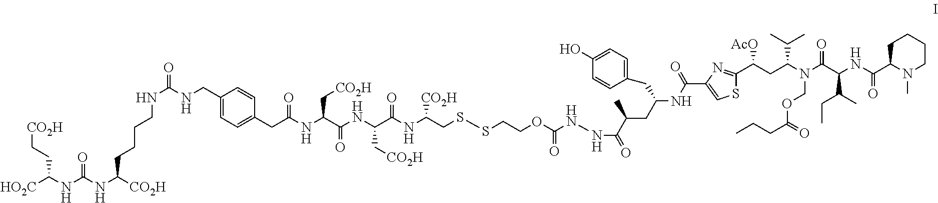 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, for treating a cancer in a patient, wherein stable disease results after the PSMA ligand-tubulysin compound I, or a pharmaceutically acceptable salt thereof, is administered. In some aspects of these embodiments, the use comprises administering to the patient a therapeutically effective amount of the PSMA ligand-tubulysin compound I.

In other embodiments, the present disclosure provides use of a PSMA ligand-tubulysin compound I

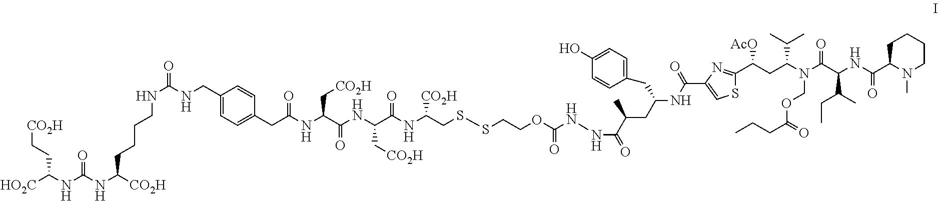 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, in the preparation of a medicament useful for the treatment of a cancer in a patient, wherein stable disease results after the PSMA ligand-tubulysin compound I, or a pharmaceutically acceptable salt thereof, is administered. In some aspects, the medicament comprises a therapeutically effective amount of the PSMA ligand-tubulysin compound I, or a pharmaceutically acceptable salt thereof.

In some aspects of these embodiments, the patient has been treated with at least one prior treatment. In some aspects of these embodiments, the at least one prior treatment is selected from the group consisting of a chemotherapeutic agent, surgery, radiation therapy, immunotherapy, photodynamic therapy, stem cell therapy, and hyperthermia. In some aspects of these embodiments, the at least one prior treatment is a systemic treatment. In some aspects of these embodiments, the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof. In some aspects of these embodiments, the cancer is a PSMA expressing cancer. In some aspects of these embodiments, the compound is at least about 98 percent pure.

In some aspects of these embodiments, the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma. In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction. In some aspects of these embodiments, the cancer is a primary or secondary brain cancer. In some aspects of these embodiments, the cancer is prostate cancer. In some aspects of these embodiments, the cancer is metastatic prostate cancer.

In some aspects of these embodiments, the PSMA ligand-tubulysin compound I, or a pharmaceutically acceptable salt thereof, is administered in a parenteral dosage form. In some aspects of these embodiments, the parenteral dosage form is selected from the group consisting of intradermal, subcutaneous, intramuscular, intraperitoneal, intravenous, and intrathecal. In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 0.1 mg/m² to about 6.0 mg/m². In some aspects of these embodiments, the therapeutically effective amount is from about 0.1 mg/m² to about 5.0 mg/m². In some aspects of these embodiments, the therapeutically effective amount is from about 0.1 mg/m² to about 4.0 mg/m². In some aspects of these embodiments, the therapeutically effective amount is from about 0.1 mg/m² to about 3.5 mg/m². In some aspects of these embodiments, the therapeutically effective amount is from about 0.1 mg/m² to about 3.0 mg/m². In some aspects of these embodiments, the therapeutically effective amount is from about 0.1 mg/m² to about 2.5 mg/m². In some aspects of these embodiments, the therapeutically effective amount is from about 0.1 mg/m² to about 2.0 mg/m².

In some aspects of these embodiments, the therapeutically effective amount is from 0.1 mg/m² to 6.0 mg/m². In some aspects of these embodiments, the therapeutically effective amount is from 0.1 mg/m² to 5.0 mg/m². In some aspects of these embodiments, the therapeutically effective amount is from 0.1 mg/m² to 4.0 mg/m². In some aspects of these embodiments, the therapeutically effective amount is from 0.1 mg/m² to 3.5 mg/m². In some aspects of these embodiments, the therapeutically effective amount is from 0.1 mg/m² to 3.0 mg/m². In some aspects of these embodiments, the therapeutically effective amount is from 0.1 mg/m² to 2.5 mg/m². In some aspects of these embodiments, the therapeutically effective amount is from 0.1 mg/m² to 2.0 mg/m².

In other embodiments, the methods and uses described herein further comprise imaging PSMA expression by the cancer. In some aspects of these embodiments, the step of imaging occurs before the step of administering. In some aspects of these embodiments, the imaging is performed by imaging wherein the imaging is selected from the group consisting of SPECT imaging, PET imaging, IHC, and FISH. In some aspects of these embodiments, the imaging is performed by SPECT imaging.

In some aspects of these embodiments, the step of imaging comprises administering to the patient a PSMA ligand-imaging conjugate of the formula II

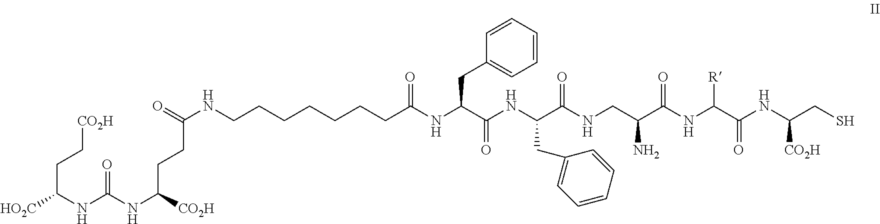

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, the step of imaging comprises administering a PSMA ligand-imaging conjugate of the formula III

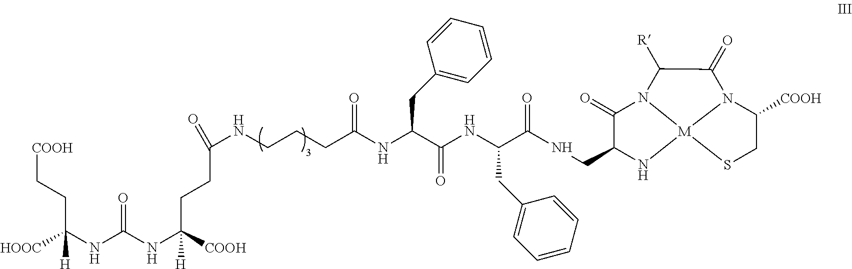

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the PSMA ligand-imaging conjugate is of the formula IIa

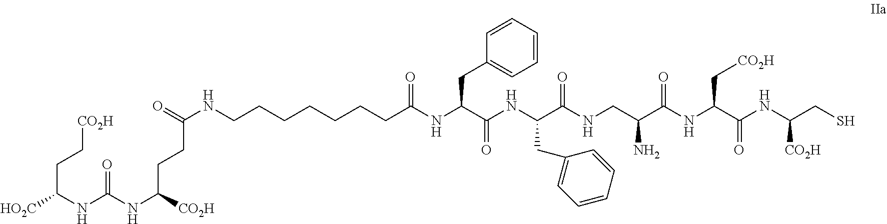 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, and wherein a radionuclide is bound to the conjugate. In some aspects of these embodiments, the PSMA ligand-imaging conjugate is of the formula IIIa

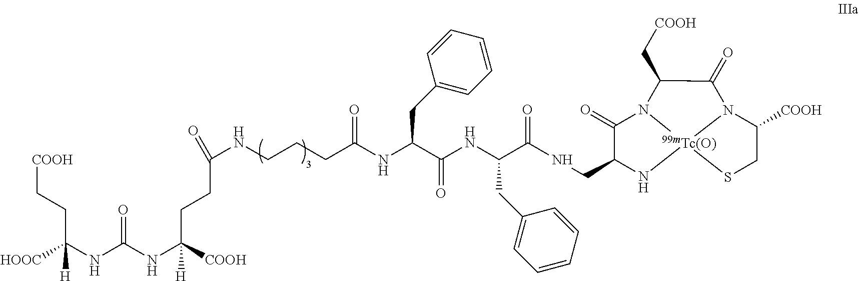

or a pharmaceutically acceptable salt thereof.

In other embodiments, the methods and uses described herein further comprise determining the PSMA status of the patient by imaging. In some aspects of these embodiments, the imaging is SPECT imaging. In some aspects of these embodiments, the PSMA status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease.

In some aspects of these embodiments, the step of determining comprises administering to the patient a PSMA ligand-imaging conjugate of the formula II

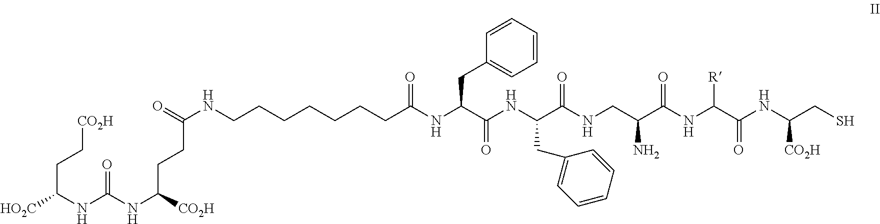

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, the step of determining comprises administering a PSMA ligand-imaging conjugate of the formula III

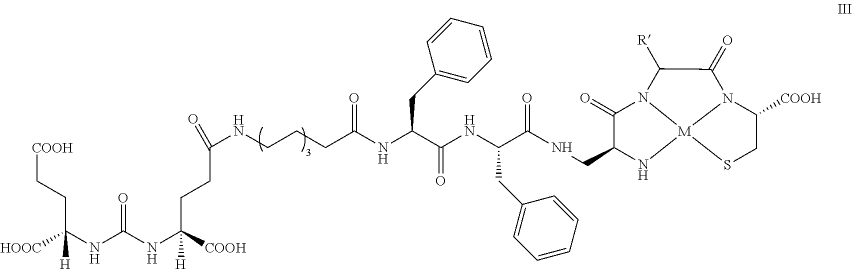

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherin M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the PSMA ligand-imaging conjugate is of the formula IIa

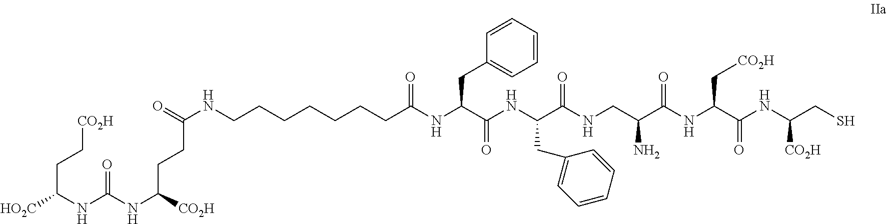 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, wherein a radionuclide is bound to the conjugate.

In some aspects of these embodiments, the PSMA ligand-imaging conjugate is of the formula IIIa

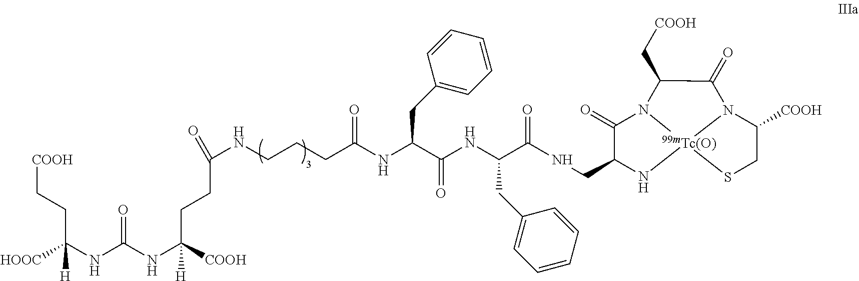

or a pharmaceutically acceptable salt thereof.

In other embodiments, the present disclosure provides a method of treating a PSMA expressing cancer in a patient in need of such treatment comprising, administering to the patient a therapeutically effective amount of a PSMA ligand-tubulysin compound I

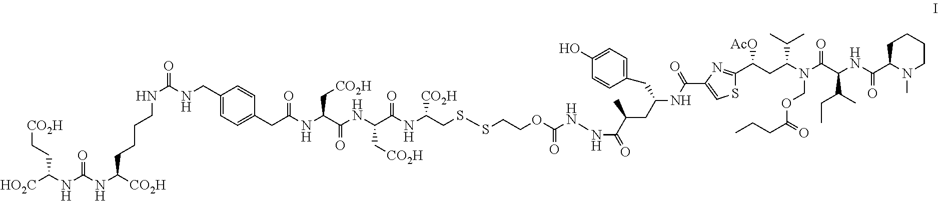 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, wherein the patient has been identified as having a PSMA expressing cancer.

In other embodiments, the present disclosure provides use of a PSMA ligand-tubulysin compound I

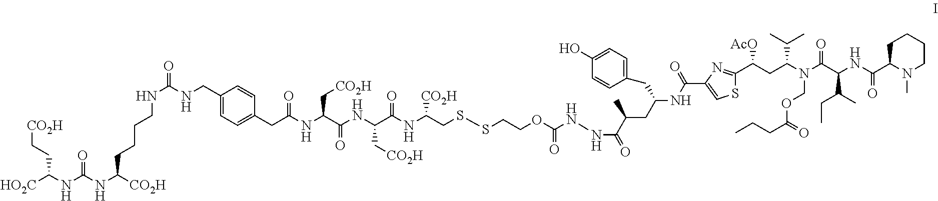 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, for treating a PSMA expressing cancer in a patient, wherein the patient has been identified as having a PSMA expressing cancer. In some aspects of these embodiments, the use comprises administering to the patient a therapeutically effective amount of a PSMA ligand-tubulysin compound I, or a pharmaceutically acceptable salt thereof.

In other embodiments, the present disclosure provides use of a PSMA ligand-tubulysin compound I

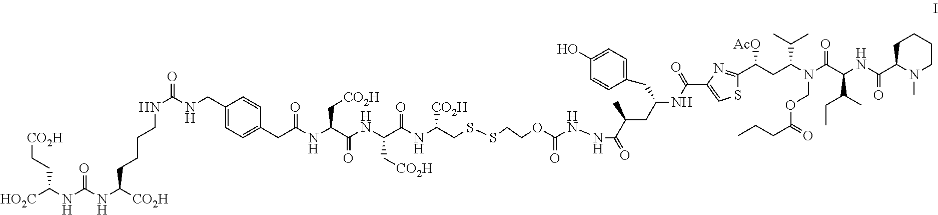 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, in the preparation of a medicament useful for the treatment of a cancer in a patient, wherein stable disease results after the PSMA ligand-tubulysin compound I, or a pharmaceutically acceptable salt thereof, is administered. In some aspects, the medicament comprises a therapeutically effective amount of the PSMA ligand-tubulysin compound I, or a pharmaceutically acceptable salt thereof.

In some aspects of these embodiments, the patient has been treated with at least one prior treatment. In some aspects of these embodiments, the at least one prior treatment is selected from the group consisting of a chemotherapeutic agent, surgery, radiation therapy, immunotherapy, photodynamic therapy, stem cell therapy, and hyperthermia. In some aspects of these embodiments, the at least one prior treatment is a systemic treatment. In some aspects of these embodiments, the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof. In some aspects of these embodiments, the cancer is a PSMA expressing cancer. In some aspects of these embodiments, the compound is at least about 98 percent pure.

In some aspects of these embodiments, the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma. In some aspects of these embodiments, the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction. In some aspects of these embodiments, the cancer is a primary or secondary brain cancer. In some aspects of these embodiments, the cancer is prostate cancer. In some aspects of these embodiments, the cancer is metastatic prostate cancer.

In some aspects of these embodiments, the PSMA ligand-tubulysin compound I, or a pharmaceutically acceptable salt thereof, is administered in a parenteral dosage form. In some aspects of these embodiments, the parenteral dosage form is selected from the group consisting of intradermal, subcutaneous, intramuscular, intraperitoneal, intravenous, and intrathecal. In some aspects of these embodiments, the therapeutically effective amount is from about 0.1 mg/m² to about 6.0 mg/m². In some aspects of these embodiments, the therapeutically effective amount is from about 0.1 mg/m² to about 5.0 mg/m². In some aspects of these embodiments, the therapeutically effective amount is from about 0.1 mg/m² to about 4.0 mg/m². In some aspects of these embodiments, the therapeutically effective amount is from about 0.1 mg/m² to about 3.5 mg/m². In some aspects of these embodiments, the therapeutically effective amount is from about 0.1 mg/m² to about 3.0 mg/m². In some aspects of these embodiments, the therapeutically effective amount is from about 0.1 mg/m² to about 2.5 mg/m². In some aspects of these embodiments, the therapeutically effective amount is from about 0.1 mg/m² to about 2.0 mg/m².

In some aspects of these embodiments, the therapeutically effective amount is from 0.1 mg/m² to 6.0 mg/m². In some aspects of these embodiments, the therapeutically effective amount is from 0.1 mg/m² to 5.0 mg/m². In some aspects of these embodiments, the therapeutically effective amount is from 0.1 mg/m² to 4.0 mg/m². In some aspects of these embodiments, the therapeutically effective amount is from 0.1 mg/m² to 3.5 mg/m². In some aspects of these embodiments, the therapeutically effective amount is from 0.1 mg/m² to 3.0 mg/m². In some aspects of these embodiments, the therapeutically effective amount is from 0.1 mg/m² to 2.5 mg/m². In some aspects of these embodiments, the therapeutically effective amount is from 0.1 mg/m² to 2.0 mg/m².

In other embodiments the methods described herein further comprise imaging PSMA expression by the cancer. In some aspects of these embodiments, the step of imaging occurs before the step of administering. In some aspects of these embodiments, the imaging is performed by imaging and the imaging is selected from the group consisting of SPECT imaging, PET imaging, IHC, and FISH. In some aspects of these embodiments, the imaging is performed by SPECT imaging.

In some aspects of these embodiments, the step of imaging comprises administering to the patient a PSMA ligand-imaging conjugate of the formula II

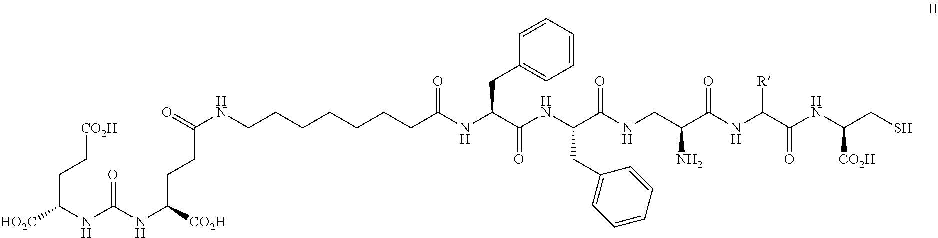

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, the step of imaging comprises administering a PSMA ligand-imaging conjugate of the formula III

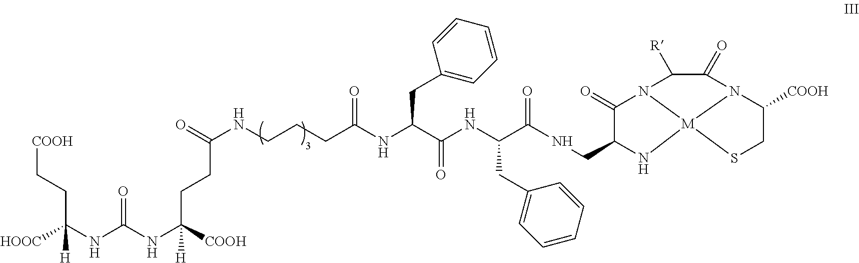

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide. In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium.

In some aspects of these embodiments, the PSMA ligand-imaging conjugate is of the formula IIa

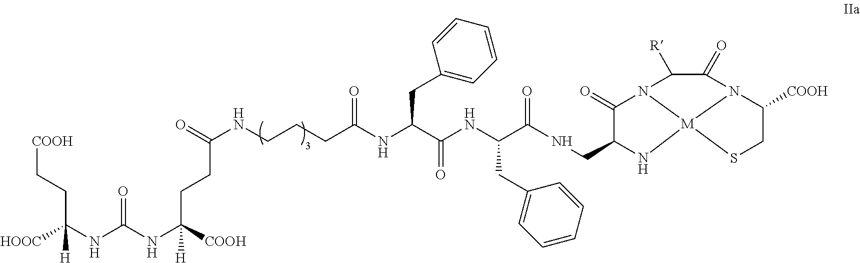

or a pharmaceutically acceptable salt thereof, wherein a radionuclide is bound to the conjugate. In some aspects of these embodiments, the PSMA ligand-imaging conjugate is of the formula IIIa

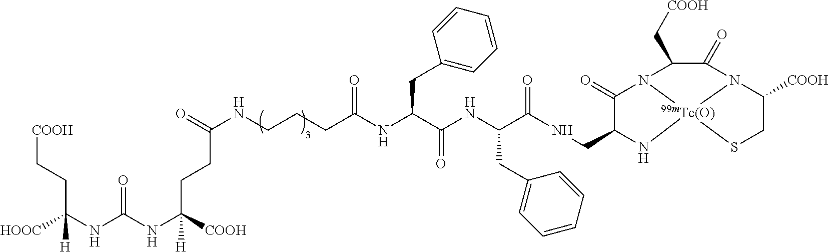

or a pharmaceutically acceptable salt thereof.

In other embodiments the methods described herein further comprise determining the PSMA status of the patient by imaging. In some aspects of these embodiments, the imaging is SPECT imaging. In some aspects of these embodiments, the PSMA status of the patient correlates with a clinical benefit to the patient. In some aspects of these embodiments, the clinical benefit is selected from the group consisting of inhibition of tumor growth, stable disease, a partial response, and a complete response. In some aspects of these embodiments, the clinical benefit is stable disease.

In some aspects of these embodiments, the step of determining comprises administering to the patient a PSMA ligand-imaging conjugate of the formula II

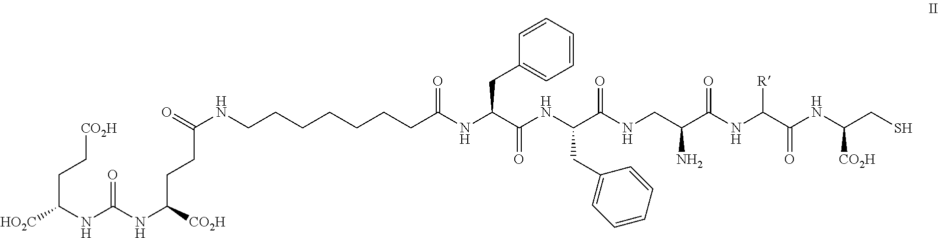

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

In some aspects of these embodiments, the step of determining comprises administering a PSMA ligand-imaging conjugate of the formula III

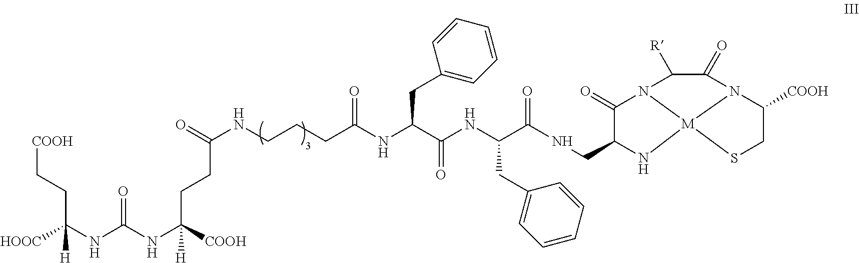

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherin M is a cation of a radionuclide.

In some aspects of these embodiments, M in the conjugate, or a pharmaceutically acceptable salt thereof, is selected from the group consisting of an isotope of gallium, an isotope of indium, an isotope of copper, an isotope of technetium, and an isotope of rhenium. In some aspects of these embodiments, M in the conjugate, or a pharmaceutically acceptable salt thereof, is an isotope of technetium. In some aspects of these embodiments, the PSMA ligand-imaging conjugate is of the formula IIa

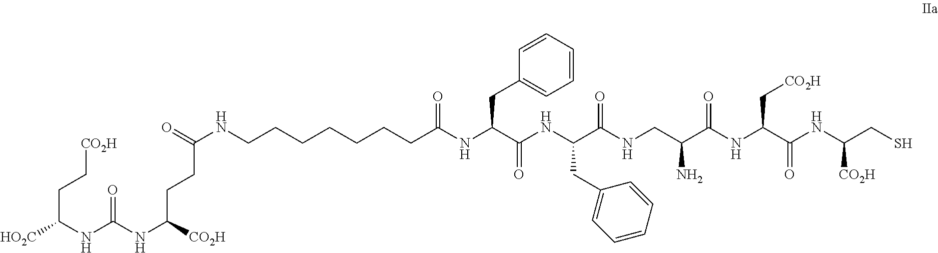

or a pharmaceutically acceptable salt thereof, and a radionuclide is bound to the conjugate.

In some aspects of these embodiments, the PSMA ligand-imaging conjugate is of the formula IIIa

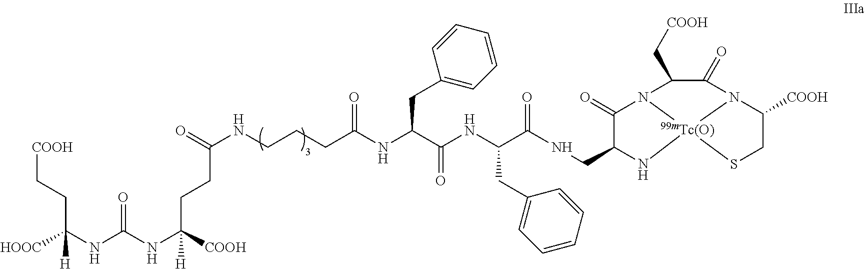

or a pharmaceutically acceptable salt thereof.

Embodiments of the invention are further described by the following enumerated clauses:

1. A method for treating a cancer in a patient in need of such treatment comprising, administering to the patient a therapeutically effective amount of a PSMA ligand-tubulysin compound I

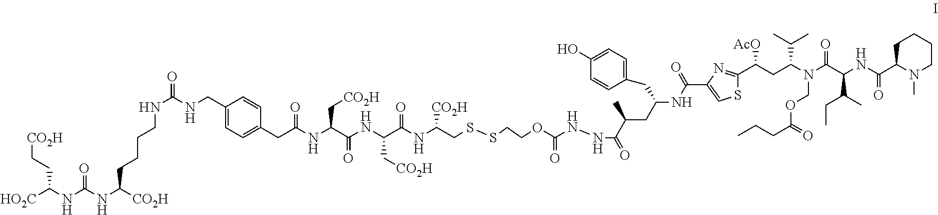 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof.

1a. Use of a PSMA ligand-tubulysin compound I

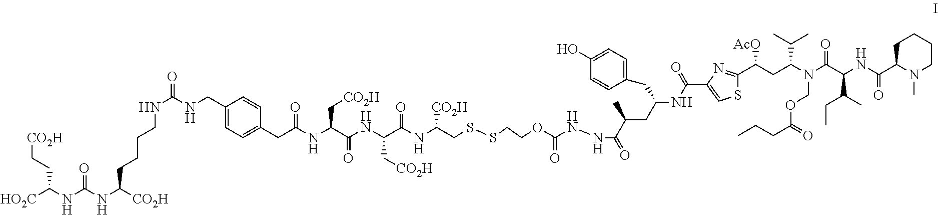 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, for treating a cancer in a patient.

1b. Use of a PSMA ligand-tubulysin compound I

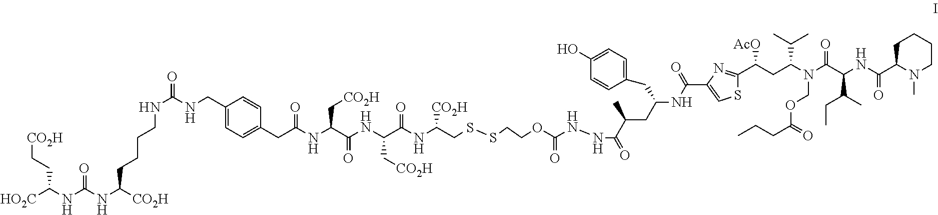 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, in the preparation of a medicament useful for the treatment of a cancer in a patient.

2. The method or use of clause 1, 1a or 1b, wherein the cancer is a PSMA expressing cancer.

3. The method or use of clause 1 or 2, wherein the PSMA ligand-tubulysin compound is at least about 98 percent pure.

4. The method or use of any one of clauses 1 to 3, wherein the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma.

5. The method or use of any one of clauses 1 to 3, wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction.

6. The method or use of any one of clauses 1 to 5, wherein the cancer is a primary or secondary brain cancer.

7. The method or use of any one of clauses 1 to 5, wherein the cancer is prostate cancer.

8. The method or use of any one of clauses 1 to 5, wherein the cancer is metastatic prostate cancer.

9. The method or use of any one of clauses 1 to 8, wherein the PSMA ligand-tubulysin compound I, or a pharmaceutically acceptable salt thereof, is administered in a parenteral dosage form.

10. The method or use of clause 9, wherein the parenteral dosage form is selected from the group consisting of intradermal, subcutaneous, intramuscular, intraperitoneal, intravenous, and intrathecal.

11. The method or use of any one of clauses 1 to 10, wherein the therapeutically effective amount is from about 0.1 mg/m² to about 6.0 mg/m².

12. The method or use of any one of clauses 1 to 11, wherein the therapeutically effective amount is from about 0.1 mg/m² to about 5.0 mg/m².

13. The method or use of any one of clauses 1 to 12, wherein the therapeutically effective amount is from about 0.1 mg/m² to about 4.0 mg/m².

14. The method or use of any one of clauses 1 to 13, wherein the therapeutically effective amount is from about 0.1 mg/m² to about 3.5 mg/m².

15. The method or use of any one of clauses 1 to 14, wherein the therapeutically effective amount is from about 0.1 mg/m² to about 3.0 mg/m².

16. The method or use of any one of clauses 1 to 15, wherein the therapeutically effective amount is from about 0.1 mg/m² to about 2.5 mg/m².

17. The method or use of any one of clauses 1 to 16, wherein the therapeutically effective amount is from about 0.1 mg/m² to about 2.0 mg/m².

18. The method or use of any one of clauses 1 to 10, wherein the therapeutically effective amount is from 0.1 mg/m² to 6.0 mg/m².

19. The method or use of any one of clauses 1 to 11, wherein the therapeutically effective amount is from 0.1 mg/m² to 5.0 mg/m².

20. The method or use of any one of clauses 1 to 12, wherein the therapeutically effective amount is from 0.1 mg/m² to 4.0 mg/m².

21. The method or use of any one of clauses 1 to 13, wherein the therapeutically effective amount is from 0.1 mg/m² to 3.5 mg/m².

22. The method or use of any one of clauses 1 to 14, wherein the therapeutically effective amount is from 0.1 mg/m² to 3.0 mg/m².

23. The method or use of any one of clauses 1 to 15, wherein the therapeutically effective amount is from 0.1 mg/m² to 2.5 mg/m².

24. The method or use of any one of clauses 1 to 16, wherein the therapeutically effective amount is from 0.1 mg/m² to 2.0 mg/m².

25. The method or use of any one of clauses 1 to 24, further comprising imaging PSMA expression by the cancer.

26. The method or use of clause 25, wherein the step of imaging occurs before the step of administering.

27. The method or use of clause 25 or 26, wherein the imaging is performed by imaging and wherein the imaging is selected from the group consisting of SPECT imaging, PET imaging, IHC, and FISH.

28. The method or use of any one of clauses 25 to 27, wherein the imaging is performed by SPECT imaging.

29. The method or use of any one of clauses 1 to 28, further comprising determining the PSMA status of the patient by imaging.

30. The method or use of clause 29, wherein the imaging is SPECT imaging.

31. The method or use of any one of clauses 29 or 30, wherein the PSMA status of the patient correlates with a clinical benefit to the patient.

32. The method or use of clause 31, wherein the clinical benefit is selected from the group consisting of inhibition of tumor growth, stable disease, a partial response, and a complete response.

33. The method or use of clause 31 or 32, wherein the clinical benefit is stable disease.

34. The method or use of any one of clauses 31 to 33, wherein at least one PSMA positive lesion indicate functionally active PSMA.

35. The method or use of any one of clauses 25 to 34, wherein the step of imaging comprises administering to the patient a PSMA ligand-imaging conjugate of the formula II

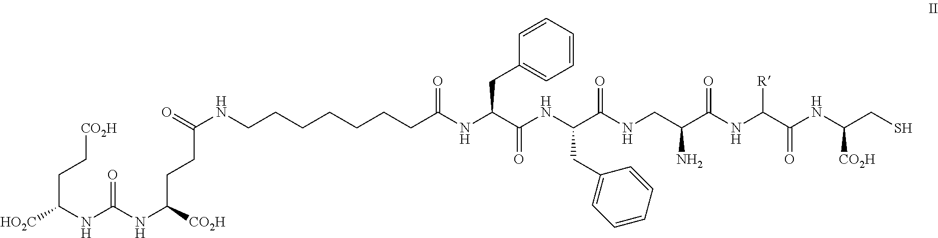

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

36. The method or use of any one of clauses 25 to 35, wherein the step of imaging comprises administering a PSMA ligand-imaging conjugate of the formula III

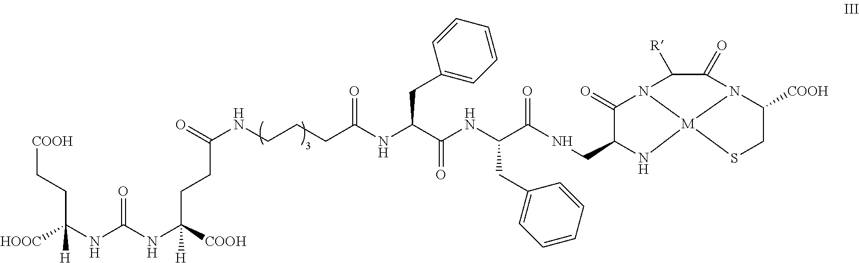

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide.

37. The method or use of clause 36, wherein M in the conjugate, or a pharmaceutically acceptable salt thereof, is selected from the group consisting of an isotope of gallium, an isotope of indium, an isotope of copper, an isotope of technetium, and an isotope of rhenium.

38. The method or use of clause 36 or 37, wherein M in the conjugate, or a pharmaceutically acceptable salt thereof, is an isotope of technetium.

39. The method or use of clause 35, wherein the PSMA ligand-imaging conjugate is of the formula IIa

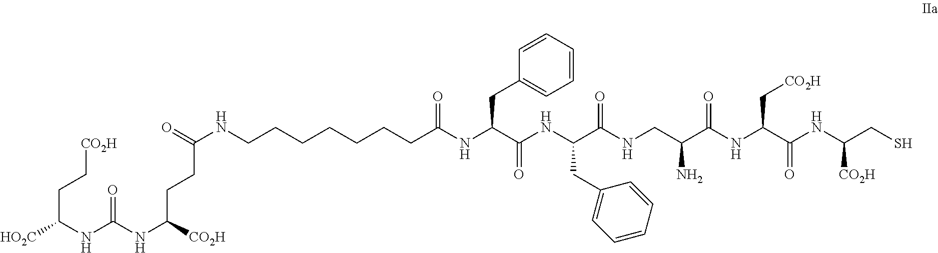

or a pharmaceutically acceptable salt thereof, and wherein a radionuclide is bound to the conjugate.

40. The method or use of any one of clauses 35 to 39, wherein the PSMA ligand-imaging conjugate is of the formula IIIa

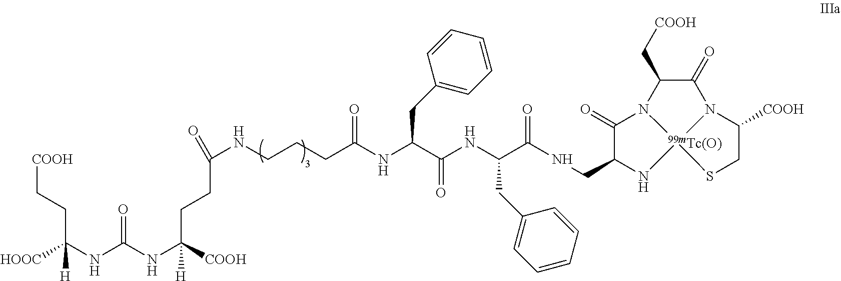

or a pharmaceutically acceptable salt thereof.

41. The method or use of any one of clauses 29 to 34, wherein the step of determining comprises administering to the patient a PSMA ligand-imaging conjugate of the formula II

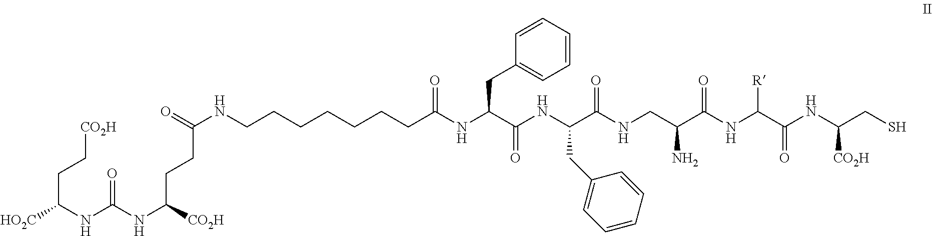

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

42. The method or use of any one of clauses 29 to 34, wherein the step of determining comprises administering a PSMA ligand-imaging conjugate of the formula III

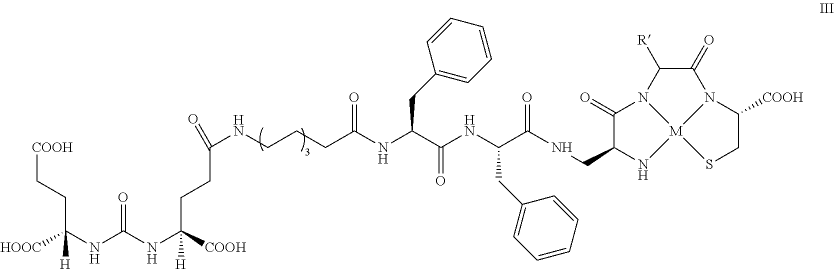

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide.

43. The method or use of clause 42, wherein M in the conjugate, or a pharmaceutically acceptable salt thereof, is selected from the group consisting of an isotope of gallium, an isotope of indium, an isotope of copper, an isotope of technetium, and an isotope of rhenium.

44. The method or use of clause 42 or 43, wherein M in the conjugate, or a pharmaceutically acceptable salt thereof, is an isotope of technetium.

45. The method or use of any one of clauses 41 to 44, wherein the PSMA ligand-imaging conjugate is of the formula IIa

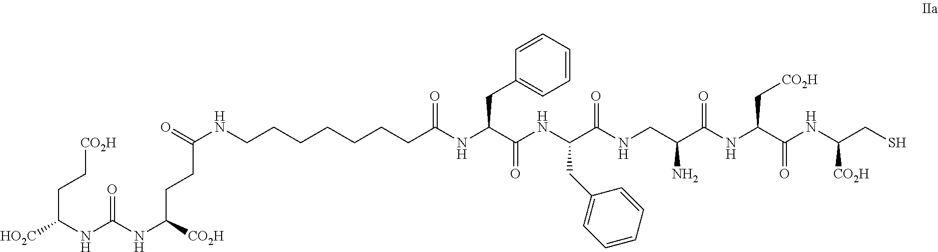 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, and wherein a radionuclide is bound to the conjugate.

46. The method or use of any one of clauses 41 to 45, wherein the PSMA ligand-imaging conjugate is of the formula IIIa

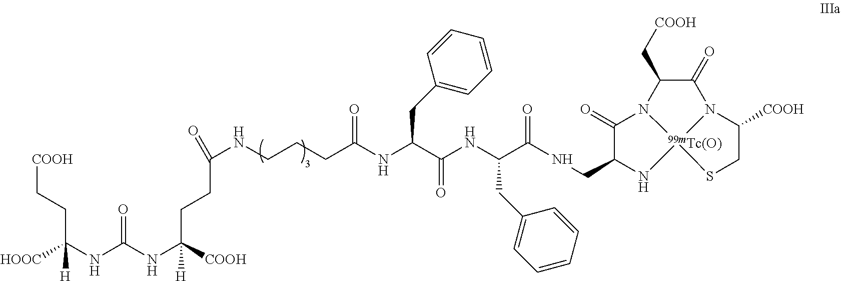

or a pharmaceutically acceptable salt thereof.

47. A method of treating a cancer in a patient in need of such treatment comprising, administering to the patient a therapeutically effective amount of a PSMA ligand-tubulysin compound I

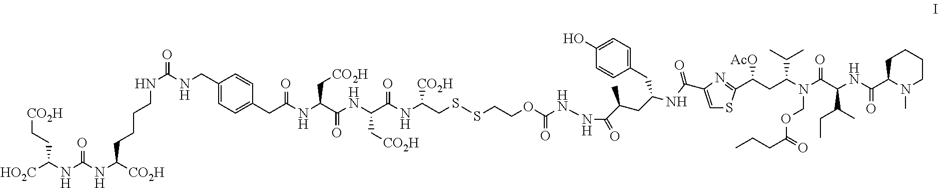 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, wherein stable disease results after the PSMA ligand-tubulysin compound I, or a pharmaceutically acceptable salt thereof, is administered.

47a. Use of a PSMA ligand-tubulysin compound I

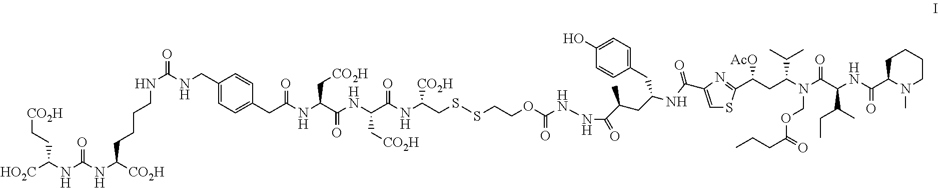 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, for treating a cancer in a patient.

47b. Use of a PSMA ligand-tubulysin compound I

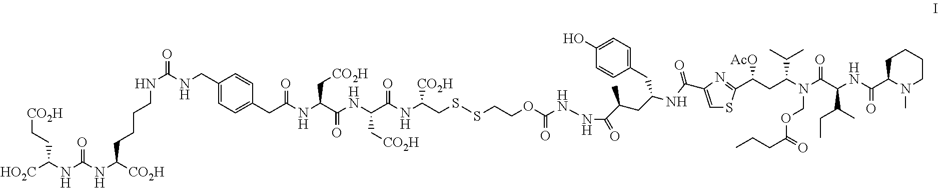 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, in the preparation of a medicament useful for the treatment of a cancer in a patient.

48. The method or use of clause 47, 47a or 47b, wherein the patient has been treated with at least one prior treatment.

49. The method or use of clause 48, wherein the at least one prior treatment is selected from the group consisting of a chemotherapeutic agent, surgery, radiation therapy, immunotherapy, photodynamic therapy, stem cell therapy, and hyperthermia.

50. The method or use of clause 48, wherein the at least one prior treatment is a systemic treatment.

51. The method or use of clause 50, wherein the systemic treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.

52. The method or use of any one of clauses 47 to 51, wherein the cancer is a PSMA expressing cancer.

53. The method or use of any one of clauses 47 to 52, wherein the compound is at least about 98 percent pure.

54. The method or use of any one of clauses 47 to 53, wherein the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma.

55. The method or use of any one of clauses 47 to 53, wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction.

56. The method or use of any one of clauses 47 to 55, wherein the cancer is a primary or secondary brain cancer.

57. The method or use of any one of clauses 47 to 55, wherein the cancer is prostate cancer.

58. The method or use of any one of clauses 47 to 55, wherein the cancer is metastatic prostate cancer.

59. The method or use of any one of clauses 47 to 58, wherein the PSMA ligand-tubulysin compound I, or a pharmaceutically acceptable salt thereof, is administered in a parenteral dosage form.

60. The method or use of clause 59, wherein the parenteral dosage form is selected from the group consisting of intradermal, subcutaneous, intramuscular, intraperitoneal, intravenous, and intrathecal.

61. The method or use of any one of clauses 47 to 60, wherein the therapeutically effective amount is from about 0.5 mg/m² to about 6.0 mg/m².

62. The method or use of any one of clauses 47 to 61, wherein the therapeutically effective amount is from about 0.5 mg/m² to about 5.0 mg/m².

63. The method or use of any one of clauses 47 to 62, wherein the therapeutically effective amount is from about 0.5 mg/m² to about 4.0 mg/m².

64. The method or use of any one of clauses 47 to 63, wherein the therapeutically effective amount is from about 0.5 mg/m² to about 3.5 mg/m².

65. The method or use of any one of clauses 47 to 64, wherein the therapeutically effective amount is from about 0.5 mg/m² to about 3.0 mg/m².

66. The method or use of any one of clauses 47 to 65, wherein the therapeutically effective amount is from about 0.5 mg/m² to about 2.5 mg/m².

67. The method or use of any one of clauses 47 to 66, wherein the therapeutically effective amount is from about 0.5 mg/m² to about 2.0 mg/m².

68. The method or use of any one of clauses 47 to 67, further comprising imaging PSMA expression by the cancer.

69. The method or use of clause 68, wherein the step of imaging occurs before the step of administering.

70. The method or use of clause 68 or 69, wherein the imaging is performed by imaging and wherein the imaging is selected from the group consisting of SPECT imaging, PET imaging, IHC, and FISH.

71. The method or use of any one of clauses 68 to 70, wherein the imaging is performed by SPECT imaging.

72. The method or use of any one of clauses 47 to 71, further comprising determining the PSMA status of the patient by imaging.

73. The method or use of clause 72, wherein the imaging is SPECT imaging.

74. The method or use of any one of clauses 71 to 73, wherein the PSMA status of the patient correlates with a clinical benefit to the patient.

75. The method or use of clause 74, wherein the clinical benefit is selected from the group consisting of inhibition of tumor growth, stable disease, a partial response, and a complete response.

76. The method or use of clause 75 wherein the clinical benefit is stable disease.

77. The method or use of any one of clauses 68 to 71, wherein the step of imaging comprises administering to the patient a PSMA ligand-imaging conjugate of the formula II

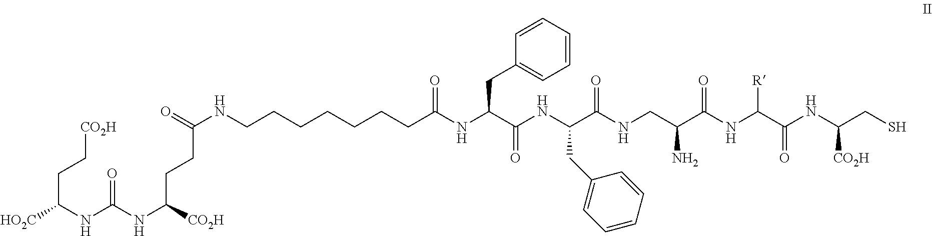

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

78. The method or use of any one of clauses 68 to 71, wherein the step of imaging comprises administering a PSMA ligand-imaging conjugate of the formula III

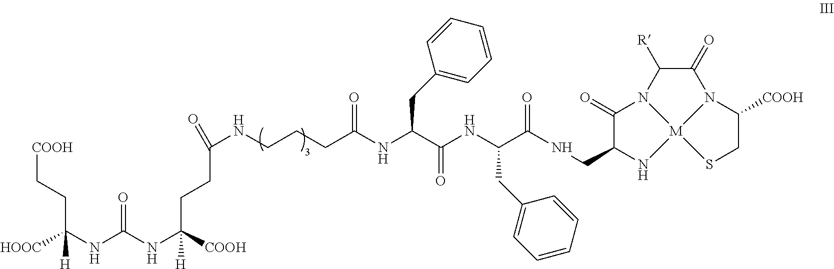

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein M is a cation of a radionuclide.

79. The method or use of clause 78, wherein M in the conjugate, or a pharmaceutically acceptable salt thereof, is selected from the group consisting of an isotope of gallium, an isotope of indium, an isotope of copper, an isotope of technetium, and an isotope of rhenium.

80. The method or use of clause 78 or 79, wherein M in the PSMA ligand-imaging conjugate, or a pharmaceutically acceptable salt thereof, is an isotope of technetium.

81. The method or use of clause 77, wherein the PSMA ligand-imaging conjugate is of the formula IIa

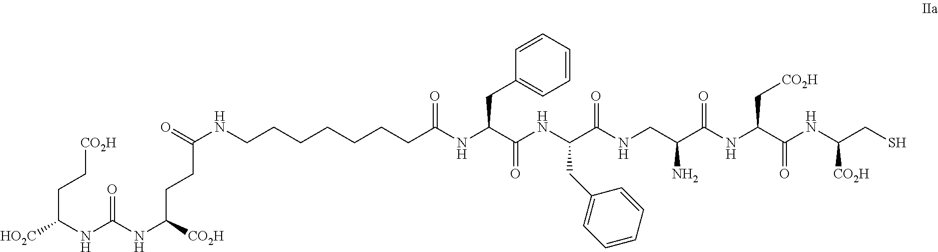 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, and wherein a radionuclide is bound to the conjugate.

82. The method or use of any one of clauses 78 to 80, wherein the PSMA ligand-imaging conjugate is of the formula IIIa

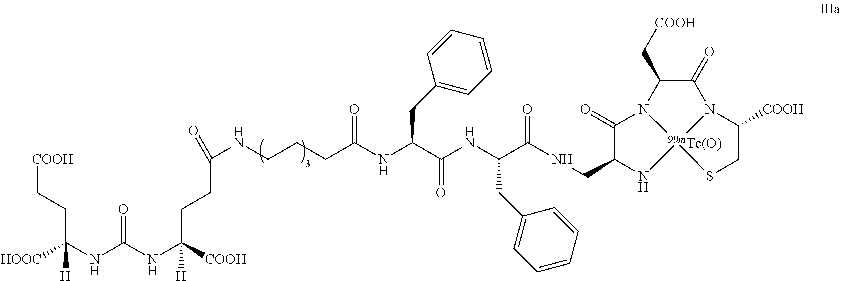

or a pharmaceutically acceptable salt thereof.

83. The method or use of any one of clauses 72 to 76, wherein the step of determining comprises administering to the patient a PSMA ligand-imaging conjugate of the formula II

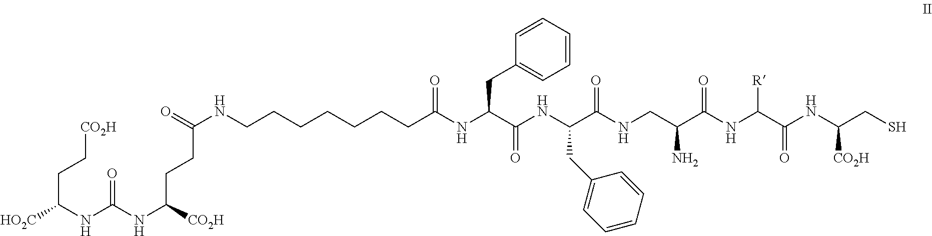

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to a conjugate.

84. The method or use of any one of clauses 72 to 76, wherein the step of determining comprises administering a PSMA ligand-imaging conjugate of the formula III

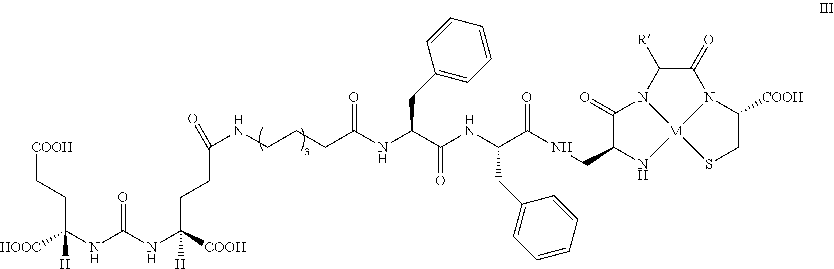

or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherin M is a cation of a radionuclide.

85. The method or use of clause 84, wherein M in the conjugate, or a pharmaceutically acceptable salt thereof, is selected from the group consisting of an isotope of gallium, an isotope of indium, an isotope of copper, an isotope of technetium, and an isotope of rhenium.

86. The method or use of clause 84 or 85, wherein M in the conjugate, or a pharmaceutically acceptable salt thereof, is an isotope of technetium.

87. The method or use of any one of clauses 84 to 86, wherein the PSMA ligand-imaging conjugate is of the formula IIa

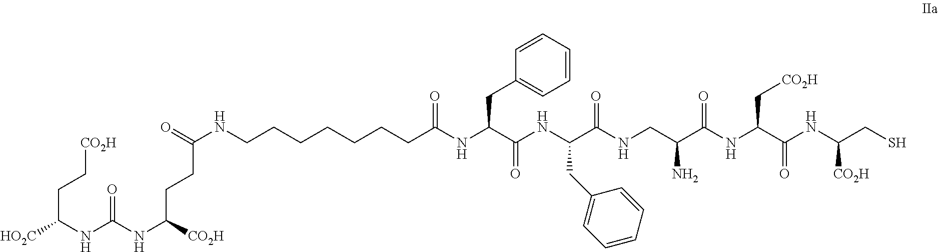 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]

or a pharmaceutically acceptable salt thereof, and wherein a radionuclide is bound to the conjugate.

88. The method or use of any one of clauses 84 to 87, wherein the PSMA ligand-imaging conjugate is of the formula IIIa

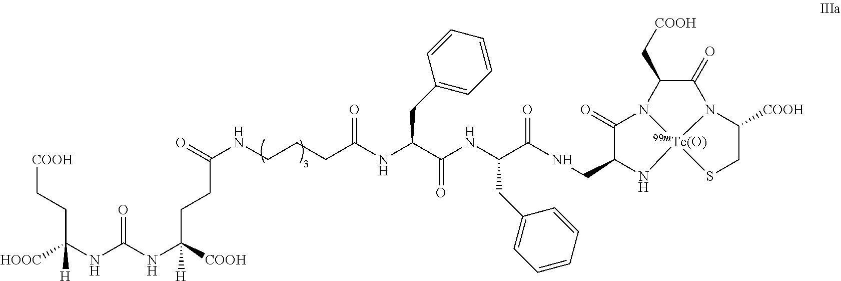

or a pharmaceutically acceptable salt thereof.

89. A method of treating a PSMA expressing cancer in a patient in need of such treatment comprising, administering to the patient a therapeutically effective amount of a PSMA ligand-tubulysin compound I

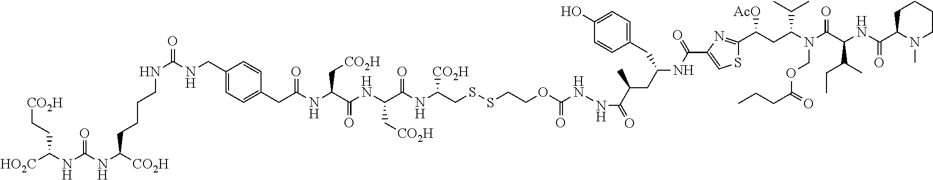 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, wherein the patient has been identified as having a PSMA expressing cancer.

89a. Use of a PSMA ligand-tubulysin compound I

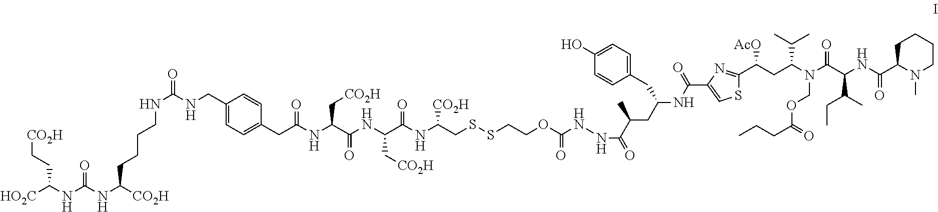 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, for treating a PSMA expressing cancer in a patient, wherein the patient has been identified as having a PSMA expressing cancer.

89b. In other embodiments, the present disclosure provides use of a PSMA ligand-tubulysin compound I

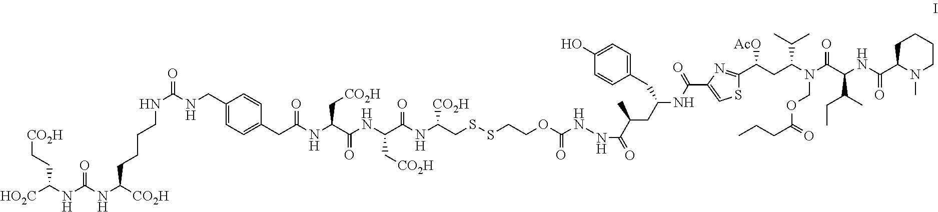 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

or a pharmaceutically acceptable salt thereof, in the preparation of a medicament useful for the treatment of a cancer in a patient, wherein stable disease results after the PSMA ligand-tubulysin compound I, or a pharmaceutically acceptable salt thereof, is administered.

### BRIEF DESCRIPTION OF THE DRAWINGS

FIG. 1A shows a PET bone scan of a tumor lesion; and

FIG. 1B shows a PSMA-imaging conjugate IIIa (^{99m}Tc-EC0652) scan of the tumor lesion shown in FIG. 1A. PSMA-imaging conjugate IIIa (^{99m}Tc-EC0652) showed good uptake in the tumor lesion.

### DEFINITIONS

In accordance with the invention, “functionally active PSMA” means a cell surface membrane-bound glycoprotein that binds to a PSMA ligand. It will be appreciated that PSMA ligands are well known to those skilled in the art such as those described in US patent publication no. US 2010/0324008 A1, incorporated herein by reference.

In accordance with the invention, “clinical benefit” means a response of a patient to treatment with PSMA ligand-tubulysin compound I where the response includes overall survival of the patient, ability to receive four or more cycles of therapy (e.g., four weeks of therapy) with PSMA ligand-tubulysin compound I, inhibition of tumor growth, stable disease, a partial response, and/or a complete response, among other clinical benefits defined by the Food and Drug Administration in the United States of America.

In accordance with the invention, “inhibition of tumor growth” means reduction in tumor size, complete disappearance of a tumor, or growth of a patient tumor of less than 30% over the course of therapy with PSMA ligand-tubulysin compound I.

In accordance with the invention, “stable disease” means no material progression of disease in a patient over the course of therapy with PSMA ligand-tubulysin compound I.

In accordance with the invention, “a partial response” means a decrease in tumor size of 30% or greater in a patient treated with PSMA ligand-tubulysin compound I.

In accordance with the invention, “a complete response” means the disappearance of detectable disease in a patient treated with PSMA ligand-tubulysin compound I.

In accordance with the invention, “prior treatment” means the patient has been treated with at least one prior treatment known in the art. It will be appreciated that a prior treatment can be any treatment known to those of skill in the art, including, but not limited, chemotherapeutic agent, surgery, radiation therapy, immunotherapy, photodynamic therapy, stem cell therapy, hyperthermia, and the like. Prior treatments can include systemic treatments including, but not limited to treatment with palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.

In accordance with the inventions, the term “alkyl” includes a chain of carbon atoms, which is optionally branched. It will be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. Illustrative alkyl groups include, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. As used herein, a “carboxyalkyl” group includes a combination of an “alkyl” group as described herein with a “carboxy” group. As used herein, a “hydroxyalkyl” group includes a combination of an “alkyl” group as described herein with a “hydroxy” group. As used herein, a “aminoalkyl” group includes a combination of an “alkyl” group as described herein with a “amino” group.

In accordance with the invention, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium.

In accordance with the invention, the term “aryl” includes monocyclic and polycyclic aromatic carbocyclic groups having from 6 to 14 ring carbon atoms, each of which may be optionally substituted. Illustrative aromatic carbocyclic groups described herein include, but are not limited to, phenyl, naphthyl, and the like. In accordance with the invention, the term “heteroaryl” includes aromatic heterocyclic groups, having from 5 to 10 ring atoms, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like. In accordance with the invention, the term “heteroarylalkyl” includes a combination of an “alkyl” group as described herein with a “heteroaryl” group described herein. In accordance with the invention, the term “arylalkyl” includes a combination of an “alkyl” group as described herein with a “aryl” group described herein, for example a benzyl group.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

In accordance with the invention, the term “administering” as used herein includes all means of introducing the PSMA ligand-tubulysin compound and PSMA ligand-imaging conjugates described herein to the patient, including, but not limited to, oral (po), intravenous (iv), intramuscular (im), subcutaneous (sc), transdermal, inhalation, buccal, ocular, sublingual, vaginal, rectal, and the like. The PSMA ligand-tubulysin compound and PSMA ligand-imaging conjugates described herein may be administered in unit dosage forms and/or formulations containing conventional nontoxic pharmaceutically-acceptable carriers, adjuvants, and vehicles.

### DETAILED DESCRIPTION

In accordance with Applicants' invention described herein, the embodiments of the numbered clauses provided in the summary above, or any combination thereof, are contemplated for combination with any of the embodiments described in the Detailed Description section of this patent application.

In one embodiment, the methods described herein can be used for both human clinical medicine and veterinary applications. Thus, a “patient” can be administered the PSMA ligand-tubulysin compound or PSMA ligand-imaging conjugates described herein, and can be human or, in the case of veterinary applications, can be a laboratory, agricultural, domestic, or wild animal. In one aspect, the patient can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In various embodiments, the cancers described herein can be a cancer cell population that is tumorigenic, including benign tumors and malignant tumors, or the cancer can be non-tumorigenic. The cancer can arise spontaneously or by such processes as mutations present in the germline of the patient or somatic mutations, or the cancer can be chemically-, virally-, or radiation-induced. Cancers applicable to the invention described herein include, but are not limited to, a glioma, a carcinoma, a sarcoma, a lymphoma, a melanoma, a mesothelioma, a nasopharyngeal carcinoma, a leukemia, an adenocarcinoma, and a myeloma.

In some aspects the cancers can be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, metastatic breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, glioma, brain stem glioma, pituitary adenoma, and adenocarcinoma of the gastroesophageal junction.

PSMA ligand-tubulysin compound I has the formula

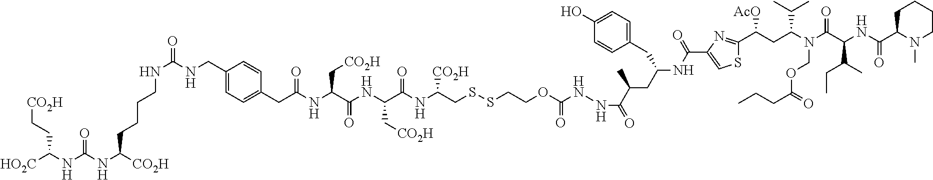 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]

and the PSMA ligand-imaging conjugates described herein include the following formulas

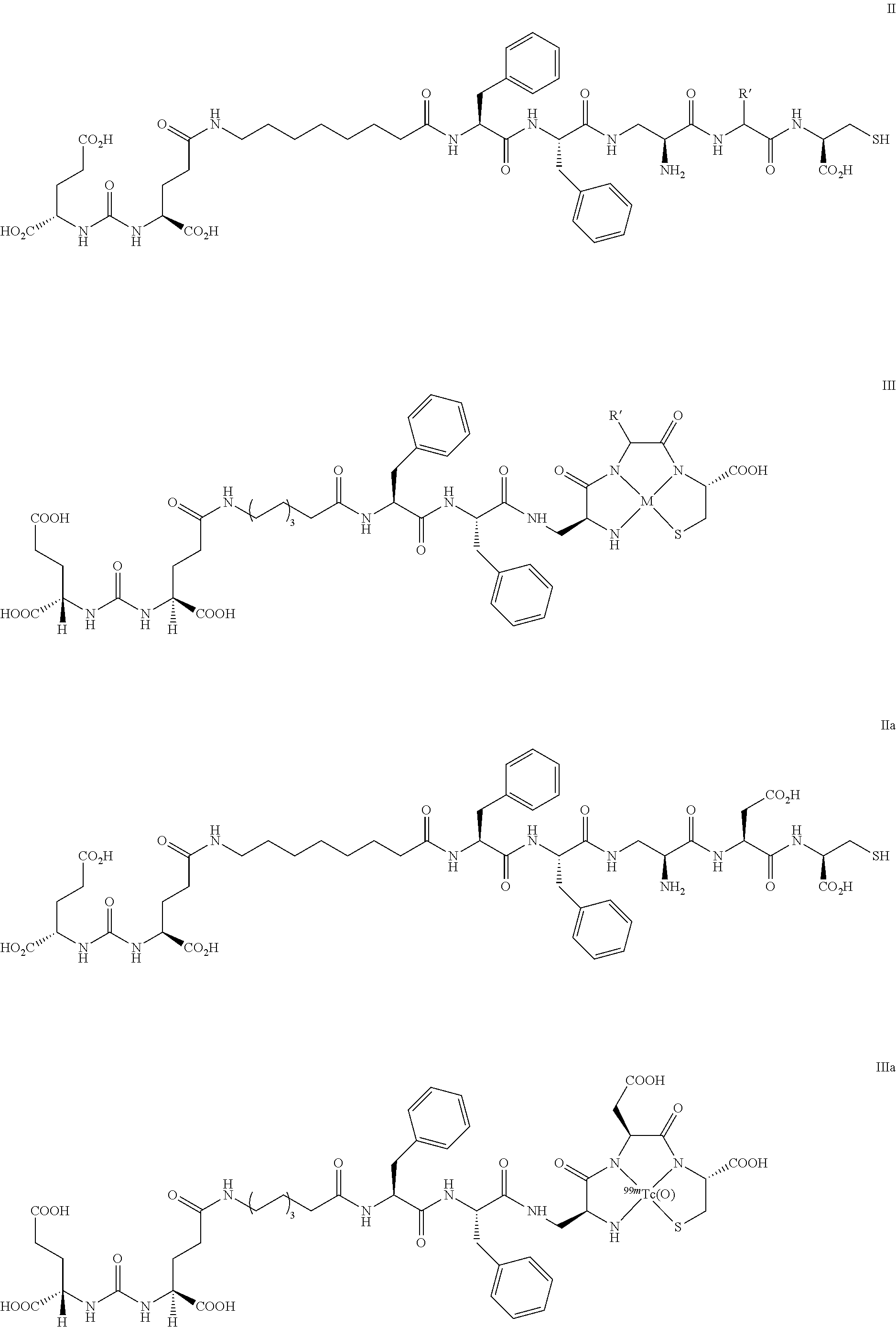

In other embodiments, any of a variety of PSMA ligand-imaging conjugates detectable by PET imaging, SPECT imaging, and the like can be used. The exact manner of imaging is not limited to the imaging agents described herein. Collectively, the PSMA ligand-imaging conjugates useful for imaging described herein, including those described by formulas and the agents useful for PET imaging, SPECT imaging, etc. are referred to as “PSMA ligand-imaging conjugates.”

In one embodiment, the PSMA ligand-tubulysin compound and PSMA ligand-imaging conjugates described herein bind to expressed PSMA on cancer cells. In one illustrative aspect, the PSMA ligand-tubulysin compound and PSMA ligand-imaging conjugates are capable of differentially binding to PSMA on cancer cells compared to normal cells due to preferential expression (or over-expression) of PSMA on the cancer cells.

In other embodiments of the methods described herein, pharmaceutically acceptable salts of the PSMA ligand-tubulysin compound and PSMA ligand-imaging conjugates described herein are provided. Pharmaceutically acceptable salts of the PSMA ligand-tubulysin compound and PSMA ligand-imaging conjugates described herein include acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the PSMA ligand-tubulysin compound and PSMA ligand-imaging conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one embodiment, the PSMA ligand-tubulysin compound and PSMA ligand-imaging conjugates described herein may be administered as a formulation in association with one or more pharmaceutically acceptable carriers. The carriers can be excipients. The choice of carrier will to a large extent depend on factors such as the particular mode of administration, the effect of the carrier on solubility and stability, and the nature of the dosage form. Pharmaceutical compositions suitable for the delivery of PSMA ligand-tubulysin compound and PSMA ligand-imaging conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of injectable compositions can be brought about by including in the composition an agent which delays absorption, for example, monostearate salts and gelatin.

Illustrative formats for oral administration include tablets, capsules, elixirs, syrups, and the like.

Depending upon the cancer type as described herein, the route of administration and/or whether the PSMA ligand-tubulysin compound and/or PSMA ligand-imaging conjugates are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, biweekly (b.i.w.), once a week, once a month, once a quarter, and the like. In each of these cases it is understood that the therapeutically effective amounts described herein correspond to the instance of administration, or alternatively to the total daily, weekly, monthly, or quarterly dose, as determined by the dosing protocol.

In one aspect, a PSMA ligand-tubulysin compound or a PSMA ligand-imaging conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the PSMA ligand-tubulysin compound or PSMA ligand-imaging conjugates described herein. The preparation of parenteral formulations under sterile conditions, for example, by lyophilization under sterile conditions, may readily be accomplished using standard pharmaceutical techniques well known to those skilled in the art. In one embodiment, the solubility of a PSMA ligand-tubulysin compound or a PSMA ligand-imaging conjugate used in the preparation of a parenteral formulation may be increased by the use of appropriate formulation techniques, such as the incorporation of solubility-enhancing agents.

In various embodiments, formulations for parenteral administration may be formulated for immediate and/or modified release. In one illustrative aspect, active agents of the invention (i.e., the PSMA ligand-tubulysin compound or PSMA ligand-imaging conjugates) may be administered in a time release formulation, for example in a composition which includes a slow release polymer. The active PSMA ligand-tubulysin compound or PSMA ligand-imaging conjugates can be prepared with carriers that will protect the PSMA ligand-tubulysin compound or PSMA ligand-imaging conjugate against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the PSMA ligand-tubulysin compound or PSMA ligand-imaging conjugates described herein or compositions comprising the PSMA ligand-tubulysin compound or PSMA ligand-imaging conjugates may be continuously administered, where appropriate.

In one embodiment, a kit is provided. If a combination of active PSMA ligand-tubulysin compound and PSMA ligand-imaging conjugates is to be administered, two or more pharmaceutical compositions may be combined in the form of a kit suitable for sequential administration or co-administration of the compositions. Such a kit comprises two or more separate pharmaceutical compositions, at least one of which contains a PSMA ligand-tubulysin compound or PSMA ligand-imaging conjugate described herein, and means for separately retaining the compositions, such as a container, divided bottle, or divided foil packet. In another embodiment, compositions comprising one or more of the PSMA ligand-tubulysin compound or PSMA ligand-imaging conjugates described herein, in containers having labels that provide instructions for use of the PSMA ligand-tubulysin compound or PSMA ligand-imaging conjugates for patient selection and/or treatment are provided.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active PSMA ligand-tubulysin compound or PSMA ligand-imaging conjugate into a sterile vehicle which contains a dispersion medium and any additional ingredients of those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof, or the ingredients may be sterile-filtered together.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering PSMA ligand-tubulysin compound I can be used. For example, PSMA ligand-tubulysin compound I can be administered as single doses, or the doses can be divided and administered as a multiple-dose daily regimen. Further, a staggered regimen, for example, one to five days per week can be used as an alternative to daily treatment, and for the purpose of the methods described herein, such intermittent or staggered daily regimen is considered to be equivalent to every day treatment and is contemplated. In one illustrative embodiment the patient is treated with multiple injections of PSMA ligand-tubulysin compound I to treat the cancer. In one embodiment, the patient is injected multiple times (preferably about 2 up to about 50 times) with PSMA ligand-tubulysin compound I, for example, at 12-72 hour intervals or at 48-72 hour intervals. Additional injections of PSMA ligand-tubulysin compound I can be administered to the patient at an interval of days or months after the initial injections(s) and the additional injections can prevent recurrence of the cancer.

Any suitable course of therapy with PSMA ligand-tubulysin compound I can be used. In one embodiment, individual doses and dosage regimens are selected to provide a total dose administered during a month of about 15 mg. In one illustrative example, PSMA ligand-tubulysin compound I is administered in a single daily dose administered five days a week, in weeks 1, 2, and 3 of each 4 week cycle, with no dose administered in week 4. In an alternative example, PSMA ligand-tubulysin compound I is administered in a single daily dose administered three days a week, of weeks 1, and 3 of each 4 week cycle, with no dose administered in weeks 2 and 4. In an alternative example, PSMA ligand-tubulysin compound I is administered biweekly on weeks 1 and 2, i.e. on days 1, 4, 8, 11, of a 3-week cycle. In an alternative example, PSMA ligand-tubulysin compound I is administered once weekly on weeks 1 and 2, i.e. days 1 and 8 of a 3-week cycle.

The unitary daily dosage of the PSMA ligand-tubulysin compound I can vary significantly depending on the patient condition, the cancer being treated, the route of administration of the PSMA ligand-tubulysin compound I and tissue distribution, and the possibility of co-usage of other therapeutic treatments, such as radiation therapy or additional drugs in combination therapies. The effective amount to be administered to a patient is based on body surface area, mass, and physician assessment of patient condition. Therapeutically effective doses (also referred to herein as “therapeutically effective amount”) can range, for example, from about 0.5 mg/m² to about 10.0 mg/m². The therapeutically effective doses described herein also include ranges of about 0.5 mg/m² to about 9.5 mg/m², about 0.5 mg/m² to about 9.0 mg/m², about 0.5 mg/m² to about 8.5 mg/m², about 0.5 mg/m² to about 8.0 mg/m², about 0.5 mg/m² to about 7.5 mg/m², about 0.5 mg/m² to about 7.0 mg/m², about 0.5 mg/m² to about 6.5 mg/m², about 0.5 mg/m² to about 6.0 mg/m², about 0.5 mg/m² to about 5.5 mg/m², about 0.5 mg/m² to about 5.0 mg/m², about 0.5 mg/m² to about 4.5 mg/m², about 0.5 mg/m² to about 4.0 mg/m², about 0.5 mg/m² to about 3.5 mg/m², about 0.5 mg/m² to about 3.0 mg/m², about 0.5 mg/m² to about 2.5 mg/m², about 0.5 mg/m² to about 2.0 mg/m², about 0.5 mg/m² to about 1.5 mg/m², about 1.0 mg/m² to about 9.5 mg/m², about 1.0 mg/m² to about 9.0 mg/m², about 1.0 mg/m² to about 8.5 mg/m², about 1.0 mg/m² to about 8.0 mg/m², about 1.0 mg/m² to about 7.5 mg/m², about 1.0 mg/m² to about 7.0 mg/m², about 1.0 mg/m² to about 6.5 mg/m², about 1.0 mg/m² to about 6.0 mg/m², about 1.0 mg/m² to about 5.5 mg/m², about 1.0 mg/m² to about 5.0 mg/m², about 1.0 mg/m² to about 4.5 mg/m², about 1.0 mg/m² to about 4.0 mg/m², about 1.0 mg/m² to about 3.5 mg/m², about 1.0 mg/m² to about 3.0 mg/m² about 1.0 mg/m² to about 2.5 mg/m², about 1.0 mg/m² to about 2.0 mg/m², and about 1.0 mg/m² to about 1.5 mg/m². One of skill in the art will readily appreciate that the therapeutically effective dose may vary within the various ranges provided above based on the factors noted above. The therapeutically effective dose for any particular patient or group of patients may be any number value between about 0.5 mg/m² and about 10.0 mg/m², including but not limited to 1.0 mg/m², 1.5, mg/m², 2.0 mg/m², 2.5 mg/m², 3.0 mg/m², 3.5 mg/m², 4.0 mg/m², 4.5 mg/m², 5.0 mg/m², 5.5 mg/m², 6.0 mg/m², 6.5 mg/m², 7.0 mg/m², 7.5 mg/m², 8.0 mg/m², 8.5 mg/m², 9.0 mg/m², 9.5 mg/m² and 10.0 mg/m². The total dose may be administered in single or divided doses and may, at the physician's discretion, fall outside of the typical range given herein.

The PSMA ligand-imaging conjugates and PSMA ligand-tubulysin compound described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The PSMA ligand-imaging conjugates and PSMA ligand-tubulysin compound described herein may be capable of existing as geometric isomers. Accordingly, it is to be understood that the present invention includes pure geometric isomers or mixtures of geometric isomers.

It is appreciated that the PSMA ligand-imaging conjugates and PSMA ligand-tubulysin compound described herein may exist in unsolvated forms as well as solvated forms, including hydrated forms. In general, the solvated forms are equivalent to unsolvated forms and are encompassed within the scope of the present invention. The PSMA ligand-imaging conjugates and PSMA ligand-tubulysin compound described herein may exist in multiple crystalline or amorphous forms. In general, all physical forms are equivalent for the uses contemplated by the present invention and are intended to be within the scope of the present invention.

In another embodiment, compositions and/or dosage forms for administration of PSMA ligand-tubulysin compound I are prepared from PSMA ligand-tubulysin compound I with a purity of at least about 90%, or about 95%, or about 96%, or about 97%, or about 98%, or about 99%, or about 99.5%. In another embodiment, compositions and or dosage forms for administration of PSMA ligand-tubulysin compound I are prepared from PSMA ligand-tubulysin compound I with a purity of at least 90%, or at least 95%, or at least 96%, or at least 97%, or at least 98%, or at least 99%, or at least 99.5%.

In another embodiment, compositions and/or dosage forms for administration of the PSMA ligand-imaging conjugate are prepared from the PSMA ligand-imaging conjugate with a purity of at least about 90%, or about 95%, or about 96%, or about 97%, or about 98%, or about 99%, or about 99.5%. In another embodiment, compositions and or dosage forms for administration of the PSMA ligand-imaging conjugate are prepared from the PSMA ligand-imaging conjugate with a purity of at least 90%, or at least 95%, or at least 97%, or at least 98%, or at least 99%, or at least 99.5%.

In another embodiment, compositions and/or dosage forms for administration of radiolabeled PSMA ligand-imaging conjugate are prepared from the PSMA ligand-imaging conjugate with a radiochemical purity of at least about 90%, or about 95%, or about 96%, or about 97%, or about 98%, or about 99%, or about 99.5%. In another embodiment, compositions and or dosage forms for administration of the PSMA ligand-imaging conjugate are prepared from the PSMA ligand-imaging conjugate with a purity of at least 90%, or at least 95%, or at least 96%, or at least 97%, or at least 98%, or at least 99%, or at least 99.5%.

The purity of PSMA ligand-tubulysin compound I or the PSMA ligand-imaging conjugates described herein may be measured using any conventional technique, including various chromatography or spectroscopic techniques, such as high pressure or high performance liquid chromatography (HPLC), nuclear magnetic resonance spectroscopy, TLC, UV absorbance spectroscopy, fluorescence spectroscopy, and the like.

In another embodiment, the PSMA ligand-tubulysin compound or PSMA ligand-imaging conjugate described herein is provided in a sterile container or package.

In one aspect, a clinical benefit of the patient to treatment with PSMA ligand-tubulysin compound I can be characterized utilizing Response Evaluation Criteria in Solid Tumors (RECIST) criteria. Illustratively, the criteria have been adapted from the original WHO Handbook (3), taking into account the measurement of the longest diameter for all target lesions: complete response, (CR)—the disappearance of all target lesions; partial response (PR)—at least a 30% decrease in the sum of the longest diameter of target lesions, taking as reference the baseline sum longest diameter; stable disease (SD)—neither sufficient shrinkage to qualify for partial response nor sufficient increase to qualify for progressive disease, taking as reference the smallest sum longest diameter since the treatment started; progressive disease (PD)—at least a 20% increase in the sum of the longest diameter of target lesions, taking as reference the smallest sum longest diameter recorded since the treatment started or the appearance of one or more new lesions. In another aspect overall disease response rate (ORR) is a clinical benefit and is calculated as the percent of patients who achieve a best response of CR or PR. Overall disease control rate (DCR) can be another clinical benefit and is calculated as the percent of patients who achieve a best response of CR, PR, or SD.

In one illustrative example overall survival is the time to death for a given patient defined as the number of days from the first day the patient received protocol treatment (C1D1) to the date of the patient's death. All events of death can be included, regardless of whether the event occurred while the patient was still taking the study drug or after the patient discontinued the study drug. If a patient has not died, then the data can be censored at the last study visit, or the last contact date, or the date the patient was last known to be alive, whichever is last.

Alternatively, a clinical benefit of the patient as a result of treatment with PSMA ligand-tubulysin compound I can be characterized as inhibition of tumor growth which can be identified in a patient through, for example, follow-up imaging of the patient's cancer after treatment with PSMA ligand-tubulysin compound I. For example, inhibition of tumor growth can be characterized by measuring the size of tumors in a patient after administration of PSMA ligand-tubulysin compound I according to any of the imaging techniques described herein, where the inhibition of tumor growth is indicated by a stable tumor size, or by a reduction in tumor size. It will be appreciated that the identification of inhibition of tumor growth can be accomplished using a variety of techniques, and is not limited to the imaging methods described herein (e.g CT, MRI, PET imaging, SPECT imaging or chest x-ray).

In one embodiment, a method is provided of determining whether PSMA ligand-tubulysin compound I is indicated for the treatment of a patient with cancer, the method comprising the step of determining the PSMA status in a patient with cancer wherein PSMA ligand-tubulysin compound I is indicated for the treatment of the patient if the PSMA status of the patient is positive.

In one embodiment, a method is provided of assessing whether PSMA ligand-tubulysin compound I is indicated for the treatment of a patient with one of the cancers described herein. The method comprises the steps of visually determining PSMA status in the patient wherein PSMA status is based on a imaging tumors that are PSMA positive in the patient, and wherein the PSMA ligand-tubulysin compound I is indicated for the treatment of the patient when the PSMA status of the patient is positive.

In the above-described embodiments, if a patient is in the group with positive PSMA status, a clinical benefit of PSMA ligand-tubulysin compound I treatment is indicated. In one embodiment, the clinical benefit to the patient can be overall survival of the patient, ability to receive four or more cycles of therapy with PSMA ligand-tubulysin compound I, inhibition of tumor growth, stable disease, a partial response of the patient to therapy, a complete response of the patient to therapy, disease control (i.e., the best result obtained is a complete response, a partial response, or stable disease), and/or overall disease response (i.e., the best result obtained is a complete response or a partial response). In one illustrative example, the clinical benefit for a patient being treated for pleural mesothelioma or adenocarcinoma (e.g. adenocarcinoma of the gastroesophageal junction) is stable disease.

In another embodiment, the methods described herein include the following examples. The examples further illustrate additional features of the various embodiments of the invention described herein. However, it is to be understood that the examples are illustrative and are not to be construed as limiting other embodiments of the invention described herein. In addition, it is appreciated that other variations of the examples are included in the various embodiments of the invention described herein.

### EXAMPLES

### 1. Preparation of PSMA Ligand-Tubulysin Compound I

PSMA ligand-tubulysin compound I was prepared according to the methods described in United States Patent Publication No. WO2014/078484, incorporated herein by reference. Specifically, PSMA ligand-tubulysin compound I is prepared according to the following procedure.

### a. Synthesis of 3-nitro-2-disulfenylethanol 2

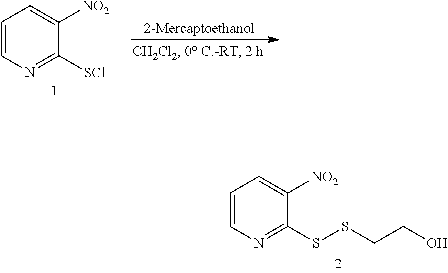

A three-necked 500 mL flask was dried and argon purged, then fitted with an addition funnel. 3-nitro-2-pyridinesulfenyl chloride 1 (5.44 g, 27.11 mmol, 1.4 equiv, available from Sigma-Aldrich) was added to the flask and dissolved in 200 mL of CH₂Cl₂. The solution was cooled to 0° C. Mercaptoethanol (1.33 mL, 18.98 mmol) was diluted with 50 mL of CH₂Cl₂ and placed in the addition funnel. The 2-mercaptoethanol solution was then added drop-wise slowly over the course of 15 minutes. The reaction progress was monitored by TLC (Rf 0.4 in 5% CH₃OH/CH₂Cl₂). Solvent was removed under reduced pressure and dried. The crude product was purified over silica gel (5% CH₃OH/CH₂Cl₂). The fractions were collected and solvent was removed by evaporating on a rotary evaporator and dried. 3.4 g of 3-nitro-2-disulfenylethanol 2 was obtained (77% yield).

### b. Synthesis of 4-nitrophenyl-(3′-nitropyridin-2′-yl)disulfenylethyl carbonate 3

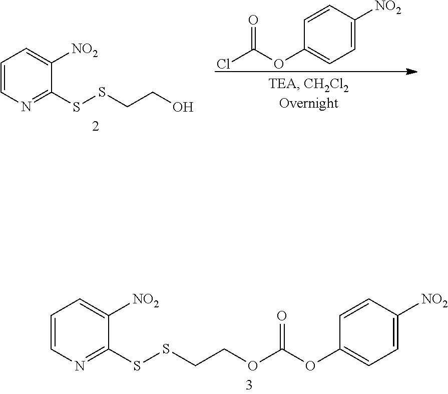 [SMILES: O=[N+]([O-])c1cccnc1SSCCO ; O=C(Cl)Oc1ccc([N+](=O)[O-])cc1 ; O=C(OCCSSc1ncccc1[N+](=O)[O-])Oc1ccc([N+](=O)[O-])cc1]

A 250 mL Round-Bottomed Flask was dried and argon purged. 3-Nitro-2-disulfenylethanol 2 (3.413 g, 14.69 mmol) was added and dissolved in 45 mL of CH₂Cl₂. 4-nitrophenylchloroformate (3.663 g, 17.63 mmol, 1.2 equiv, available from Sigma-Aldrich) was added, along with triethylamine (2.9 mL, 20.57 mmol, 1.4 equiv), and the mixture stirred under argon overnight. The mixture was concentrated under reduced pressure and dried. The residue was purified by silica (30% EtOAc/petroleum ether) and the fractions were collected, solvent was removed under reduced pressure, and dried. 2.7 g of 4-nitrophenyl-(3′-nitropyridin-2′-yl)disulfenylethyl carbonate 3 was obtained (47% yield).

### c. Synthesis of 2-(Boc-tubutyrosine (Tut))hydrazinecarboxylic acid (3′nitropyridyl-2′-yl)disulfanylethyl ester 6

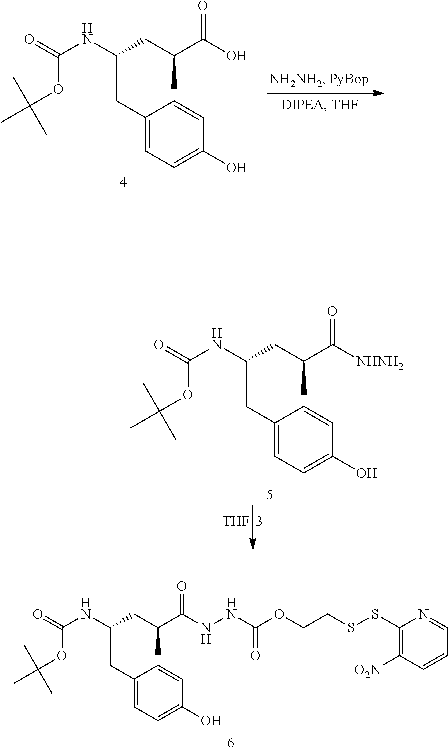 [SMILES: C[C@@H](C[C@H](Cc1ccc(O)cc1)NC(=O)OC(C)(C)C)C(=O)O ; C[C@@H](C[C@H](Cc1ccc(O)cc1)NC(=O)OC(C)(C)C)C(=O)NN ; C[C@@H](C[C@H](Cc1ccc(O)cc1)NC(=O)OC(C)(C)C)C(=O)NNC(=O)OCCSSc1ncccc1[N+](=O)[O-] ; "THF"]

10.67 g (33 mmol) of Boc-Tut-acid 4 (prepared according to the methods described in Pando, O., et. al., “First Total Synthesis of Tubulysin B”, Org. Lett. v. 11, No. 24. 5567-5569 (2009)) was dissolved in 100 mL anhydrous THF, 17.24 g (33 mmol) of PyBop, and 17.50 mL (99 mmol, 3.0 equiv) of DIPEA were added. The reaction mixture stirred for few minutes, 1.0 mL (31.68 mmol, 0.96 equiv) of hydrazine was added and stirred for 15 minutes. LC-MS analysis (X-Bridge shield RP18, 3.5 μm column; gradient 10% to 100% acetonitrile in 6 min, pH 7.4 buffer) confirmed the hydrazide 5 formation. 14.47 g (36.3 mmol, 1.1 equiv) of 4-nitrophenyl-(3′-nitropyridin-2′-yl)disulfenylethyl carbonate 2 was added. The resulting clear solution was stirred at room temperature for 24 hours. LC-MS analysis (X-Bridge shield RP18, 3.5 μm column; gradient 30% to 100% acetonitrile in 9 min, pH 7.4 buffer) indicated >98% conversion. The reaction mixture was diluted with EtOAc (˜1.0 L), washed with sat. NH₄Cl (400 mL), sat. NaHCO₃ solution (3×300 mL), and brine (300 mL). The organic layer was dried over Na₂SO₄ (100 g), and concentrated under reduced pressure. The crude product was loaded onto a Teledyne Redisep Gold Silica Column and eluted with MeOH/CH₂Cl₂ (330 g column; 0 to 10% gradient) using a CombiFlash chromatography system. The fractions were collected and solvent was removed under reduced pressure and dried. 16.10 g of 2-(Boc-Tut)hydrazinecarboxylic acid (3′nitropyridyl-2′-yl)disulfanylethyl ester 6 was obtained (82% yield).

### d. Synthesis of azido methylbutyrate dipeptide 9

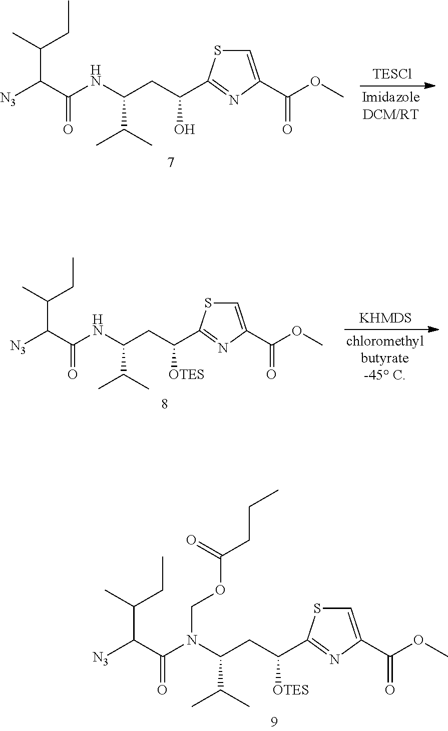

10.83 g of dipeptide 7 (27.25 mmol) (prepared according to the methods described in Peltier, H. M., et al., “The Total Synthesis of Tubulysin D”, J. Am. Chem. Soc., v. 128, pp. 16018-16019 (2006)) was dissolved in 100 mL dichloromethane and imidazole (2.05 g, 1.1 eq.) was added. The reaction mixture was stirred at room temperature to dissolve all solids and cooled in the ice bath for 10 min. TESCl (4.8 mL, 1.05 eqiv.) was added drop-wise at 0° C., stirred under argon, and warmed to room temperature over 1.5 h. TLC (3:1 hexanes/EtOAc) showed complete conversion. The reaction was filtered to remove the imidazole HCl salt. 125 mL dichloromethane was added to the filtrate, and the resulting solution was extracted with 250 mL brine. The brine layer was extracted with 125 mL dichloromethane. The combined organic phase was washed with 250 mL brine, separated, dried over 45.2 g of Na₂SO₄, and filtered. The resulting solution was concentrated under reduced pressure, co-evaporated with toluene (2×5 mL) and dried over high-vacuum overnight to give 14.96 g of crude product 8.

The crude product 8 was used without further purification. TES protected dipeptide was dissolved in 100 mL THF (anhydrous, inhibitor-free), cooled to −45° C., and stirred at −45° C. for 15 minutes before adding KHMDS (0.5 M in toluene, 61 mL, 1.05 equiv.), drop-wise. After the addition of KHMDS was finished, the reaction was stirred at −45° C. for 20 minutes, and chloromethyl butyrate (4.4 mL, 1.1 equiv.) was added. The reaction mixture was stirred at −45° C. for another 20 minutes. The reaction was quenched with 25 mL MeOH and warmed to room temperature. 250 mL EtOAc and 250 mL brine were added to the reaction mixture, and the organic phase was separated. The solvent was evaporated to reduce the volume of solution. The solution was passed through 76.5 g silica in a 350 mL sintered glass funnel. The silica plug was washed with 500 mL EtOAc/petroleum ether (1:4). The filtrate and the wash were concentrated to oily residue and dried under high vacuum to give 16.5 g product 9 as a light yellow wax.

### e. Synthesis of tripeptide methyl ester 10

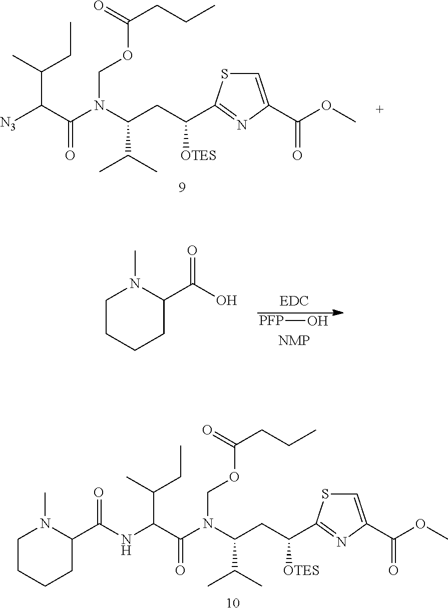

16.5 g of alkylated dipeptide 9 (26.97 mmol.), N-methyl pipecolinate (MEP) (5.51 g, 1.4 equiv.) and pentafluorophenol (7.63 g, 1.5 equiv.) were added to a 300 mL hydrogenation flask. NMP (115 mL) was then added, followed by EDC (7.78 g, 1.5 equiv.). The mixture was stirred at room temperature for overnight. 16.5 g of alkylated dipeptide 9 was dissolved in 16.5 mL NMP, transferred the solution into the hydrogenation flask, washed the residual 9 with 8 mL NMP, and transferred into the hydrogenation flask. Dry 10% Pd/C (1.45, 0.05 eq.) was added. The reaction mixture was vacuumed/back filled with hydrogen 3 times, and the flask was shaken under hydrogen (˜35 psi) for 3.5 hours. The reaction mixture was analyzed by HPLC. The reaction mixture was filtered through 40 g of celite in a 350 mL sintered glass funnel and washed with 250 mL of EtOAc. The filtrate and the wash were transferred to a separatory funnel and washed with a 1% NaHCO₃/10% NaCl solution (200 mL×3). The organic layer was isolated and dried over 45.2 g of Na₂SO₄. The solution was filtered and rotovaped under reduced pressure. A sticky amber residue was obtained and dried under high vacuum overnight to give 19.3 g of crude product. The crude product was dissolved in 10 mL of dichloromethane, split into two portions, and purified with a 330 g Teledyne Redisep Silica Gold column. The combined fractions of two purifications were evaporated and dried under high vacuum to give 7.64 g of 10 as a pale yellow solid (overall yield: 39% over 3 steps from compound 7).

### f. Synthesis of tripeptide acid 11

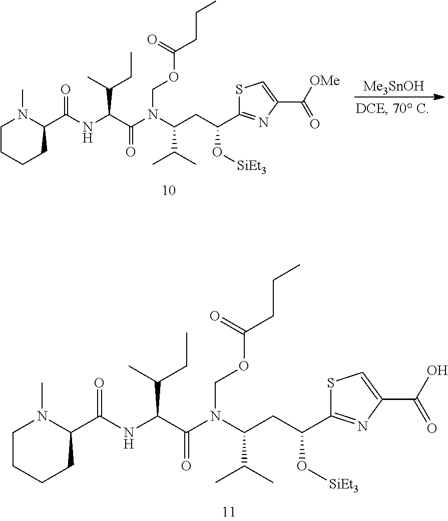

Methyl ester 10 (6.9 g, 9.7 mmol) was dissolved in 1,2-dichloroethane (193 mL) and added to a round bottomed flask, equipped with a stir bar and condenser. To this solution was added trimethyltin hydroxide (24.6 g, 14 eq.). The mixture was heated at 70° C. for 5 hours. LC-MS analysis indicated that the desired product had been formed and <15% of starting methyl ester 10 remained. The reaction was cooled in an ice bath for 30 minutes. The resulting precipitate was then removed by filtration. The filtrate was stored overnight at −20° C. The filtrate was then divided into two portions and each was subjected the chromatography procedure which follows.

Each portion was concentrated under reduced pressure and then placed under high vacuum for 30 min. The concentrate was then immediately dissolved in acetonitrile (95 mL). To this solution was then added an ammonium bicarbonate solution (95 mL; 50 mM, pH=7). This solution was loaded onto a Biotage SNAP C18 reverse phase cartridge (400 g, KP-C18-HS) and eluted with 50 mM ammonium bicarbonate and acetonitrile (1:1 to 100% ACN) using a Biotage chromatography system. Fractions were analyzed by LC-MS. Pure fractions were combined and ACN was removed under reduced pressure. The resulting aqueous suspension was extracted with EtOAc (3×). The combined organic layers were washed with brine, dried over anhydrous Na₂SO₄, and concentrated under reduced pressure. Purification of the two portions resulted in the recovery of clean 11 (4.6 g, 65%).

### g. Synthesis of acetyl tripeptide acid 13

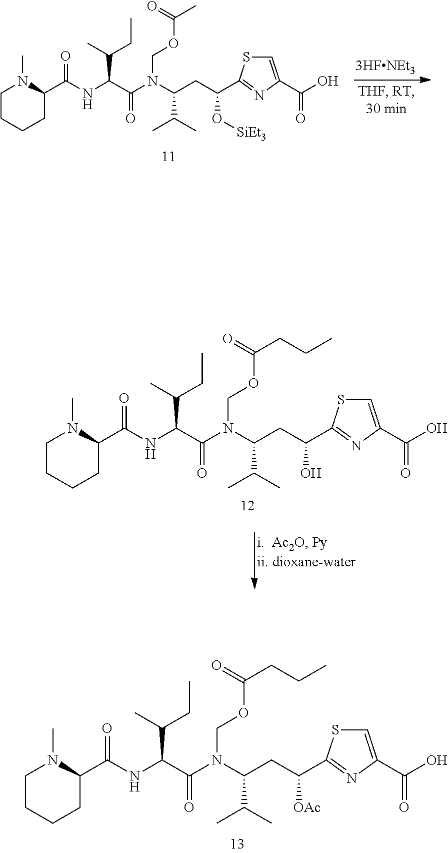

In a round bottomed flask, tripeptide acid 11 (3.9 g, 5.6 mmol) was dissolved in anhydrous THF (23 mL). To this solution was added 3 HF.TEA complex (1.8 mL, 2 eq.). The reaction was stirred at room temperature for 1 hour. LC-MS analysis indicated complete conversion to the desired des-TES product 12. The solvent was removed under reduced pressure and the residue was placed on the high vacuum for 40 minutes. The resulting residue was then dissolved in pyridine (26 mL), and acetic anhydride (7.9 mL, 15 eq.) and DMAP (25 mg) were added. The reaction was stirred at room temperature for 1 hour. LC-MS analysis indicated complete conversion to the desired acetyl tripeptide acid 13. To the reaction mixture was then added a 1:1 solution of 1,4-dioxane/water (150 mL). The reaction was stirred for 1 hour at which point the solvents were removed under high vacuum rotovap. To the residue was added toluene and the solvent was removed under vacuum (80 mL, 3×). The resulting crude 13 was dried under high vacuum overnight. The crude material was then dissolved in ACN (72 mL). Sodium phosphate buffer (50 mM, pH=7.8, 288 mL) was then added, and the pH of the resulting suspension was adjusted to neutral using saturated sodium bicarbonate solution. This solution was loaded onto a Biotage SNAP C18 reverse phase cartridge (400 g, KP-C18-HS) and eluted with water and acetonitrile (20% ACN to 65% ACN) using a Biotage chromatography system. Fractions were analyzed by LC-MS. Clean fractions were combined, the ACN was removed, and the aqueous solution was placed on the freeze dryer, resulting in purified acetyl tripeptide 13 (2.5 g, 71%).

### h. Synthesis of 2-(tubulysin B)hydrazinecarboxylic acid (3′nitropyridyl-2′-yl)disulfanylethyl ester 16

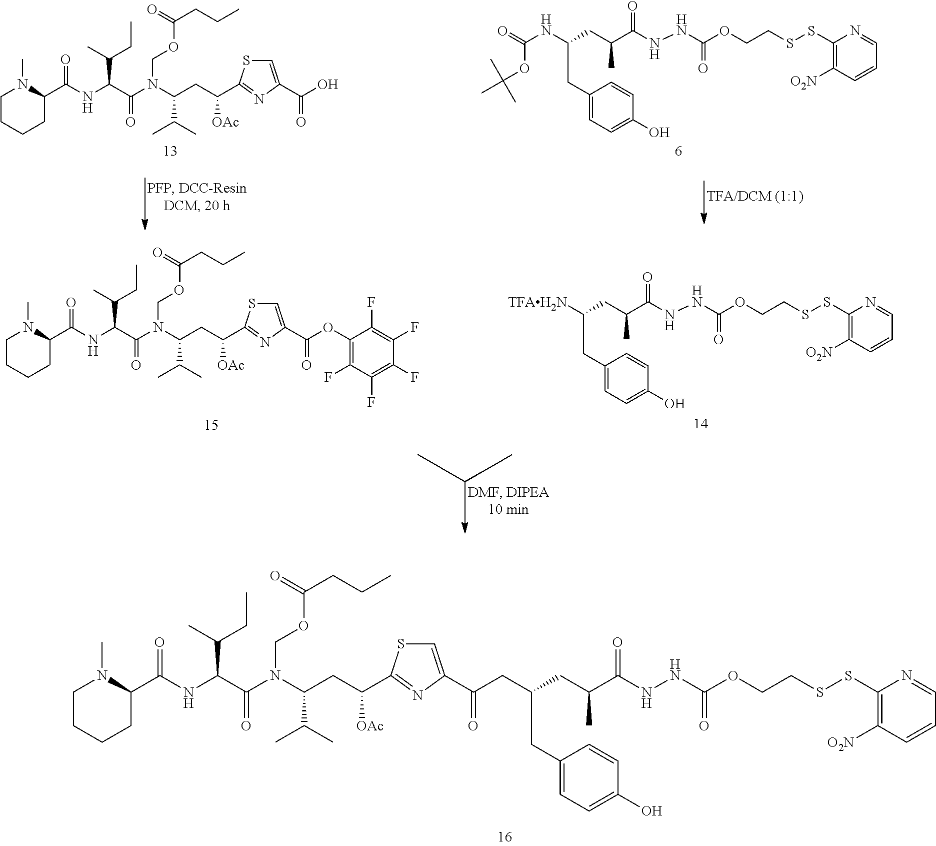

The activated Boc-Tut-fragment 6 (2.63 g, 4.42 mmol, 1.1 equiv) was treated with TFA/CH₂Cl₂ (42 mL; 1:1) and stirred for 30 minutes. LC-MS analysis (X-Bridge shield RP18, 3.5 μm column; gradient 10% to 100% acetonitrile in 6 min, pH 7.4 buffer) confirmed the product formation. TFA was removed under reduced pressure, co-evaporated with CH₂Cl₂ (3×30 mL) and activated Tut-derivative 14 was dried under high vacuum for 18 h. In another flask, the tripeptide acid 13 (2.51 g, 4.02 mmol) was dissolved in 70 mL CH₂Cl₂ (anhydrous) and 1.48 g (8.04 mmol, 2.0 equiv) of pentafluorophenol in 5 mL of CH₂Cl₂ was added, followed by 8.74 g (20.1 mmol, 5.0 equiv) of DCC-resin. The resulting reaction mixture was stirred at room temperature for 20 hours. LC-MS analysis (X-Bridge shield RP18, 3.5 μm column; gradient 10% to 100% acetonitrile in 6 min, pH 7.4 buffer) indicated >99% conversion. The DCC-resin was filtered off, the CH₂Cl₂ was removed under reduced pressure, and the pentafluorophenol activated product 15 was dried under high vacuum for 10 minutes. The residue was dissolved in 16.7 mL DMF, and DIPEA (12.6 mL, 72.36 mmol, 18.0 equiv) was added. Tut-fragment trifluoroacetic acid salt 14 in DMF (8.5 mL) was added slowly over 5 min. The resulting clear solution was stirred at room temperature for 1 h. LC-MS analysis (X-Bridge shield RP18, 3.5 μm column; gradient 10% to 100% acetonitrile in 6 min, pH 7.4 buffer) confirmed the product formation. The reaction mixture was diluted with EtOAc (700 mL), washed with brine (300 mL, 2×100 mL), dried over Na₂SO₄ (75 g), concentrated, and dried for 15 hours. The crude product was dissolved in CH₂Cl₂ (25 mL) and loaded onto a Teledyne Redisep Gold Silica Column and eluted with MeOH/CH₂Cl₂ (330 g column; 0 to 5% gradient) using Combiflash chromatographic system. The fractions were collected and solvent was removed by evaporating on a rotary evaporator and dried. 3.91 g of 2-(tubulysin B)hydrazinecarboxylic acid (3′nitropyridyl-2′-yl)disulfanylethyl ester 16 was obtained (89% yield).

### i. Preparation of Compound 105

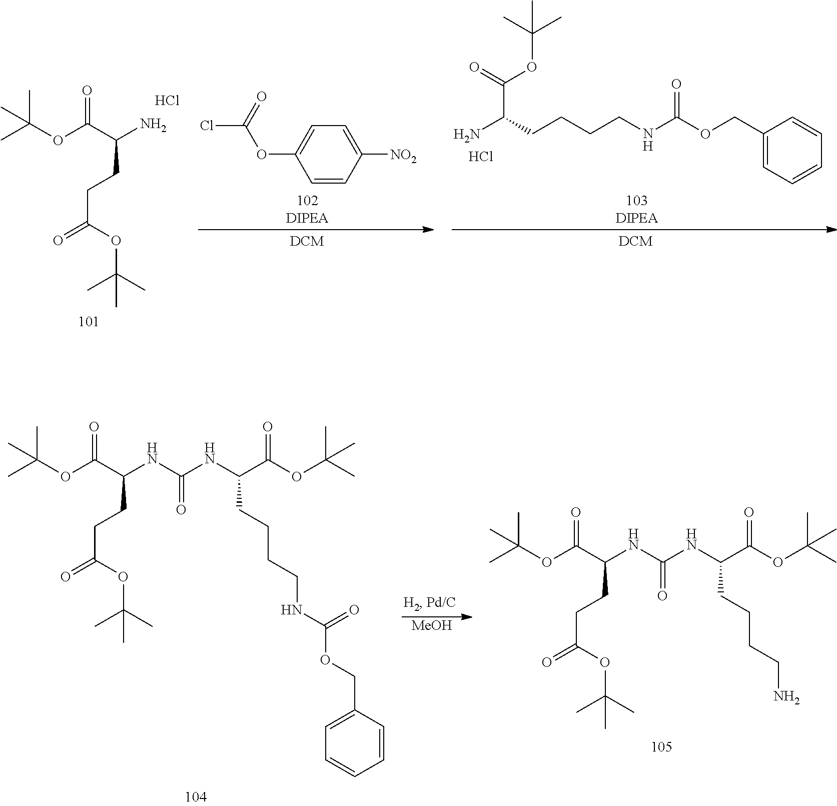

In a 250 mL round-bottom flask, H-Glu(OtBu)-OtBu.HCl (101) (4.83 g, 16.3 mmol, available from Sigma-Aldrich) and 4-nitrophenyl chloroformate (102) (3.47 g, 17.2 mmol, available from Sigma-Aldrich) were dissolved in dichloromethane (50 mL) and stirred in an ice bath under argon. Diisopropylethylamine (6.28 mL, 36.1 mmol) was added slowly, dropwise and the reaction mixture was stirred in the ice bath for 5 min, then warmed to room temperature and stirred for 30 min. H-Lys(Z)-OtBu.HCl (103) (7.01 g, 18.8 mmol) was added portionwise, followed by dropwise addition of diisopropylethylamine (6.54 mL, 37.5 mmol), and stirred at room temperature for 1 hr. The reaction mixture was concentrated under reduced pressure, then purified by silica gel chromatography in 10-100% ethyl acetate/petroleum ether to yield 104 (8.76 g, 86%, ESI m/z=622.54 [M+H]⁺).

Compound 104 (8.76 g, 14.1 mmol) was dissolved in anhydrous methanol (100 mL) and added slowly along the walls of the 250 mL round-bottom flask containing palladium on carbon, 10 wt. % (100 mg). A balloon containing hydrogen gas was attached to the flask using a three-way stopcock adapter, and the atmosphere of the flask was evacuated under reduced pressure, then replaced with hydrogen gas (3×), then stirred at room temperature under hydrogen gas for 1 hr. To the reaction mixture was added dry, untreated celite (˜20 g) and stirred for 5 min. The reaction mixture was filtered and concentrated under reduced pressure to yield 105 (6.86 g, quantitative, ESI m/z=488.46 [M+H]⁺).

### j. Preparation of Compound 107

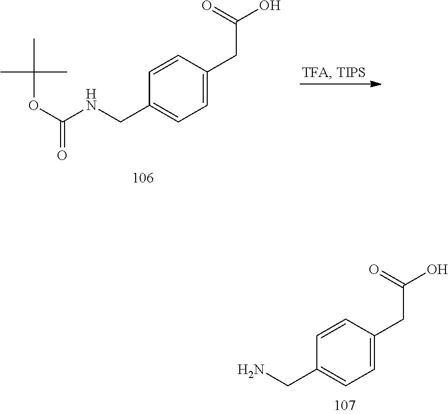 [SMILES: CC(C)(C)OC(=O)NCc1ccc(CC(=O)O)cc1 ; NCc1ccc(CC(=O)O)cc1]

Boc-4-aminomethylphenylacetic acid (106) (2.00 g, 7.5 mmol, available from VWR) dissolved in a solution of trifluoroacetic acid (9.75 mL) and triisopropylsilane (0.25 mL) and stirred at room temperature for 30 min, then concentrated under reduced pressure and coevaporated with dichloromethane (3×), then placed under vacuum, to yield 4-aminomethylphenylacetic acid (107) (quantitative yield).

### k. Preparation of Compound 108

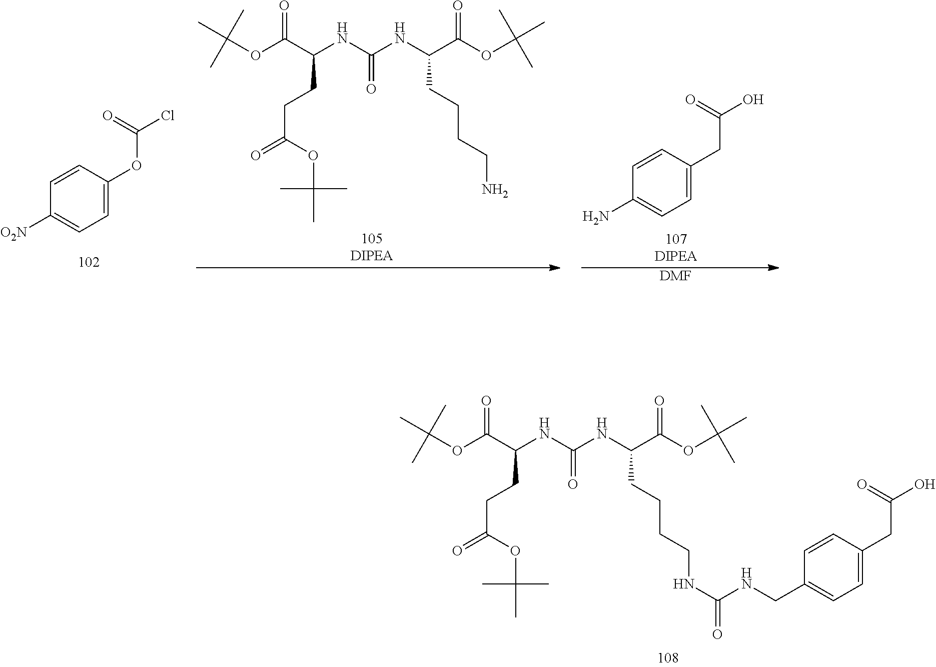

To a stirring solution of 4-nitrophenyl chloroformate (102) (1.01 g, 5.0 mmol, available from Sigma-Aldrich) in dry dimethylformamide (10 mL) was added slowly dropwise a solution of 105 (2.45 g, 5.0 mmol) and diisopropylethylamine (0.88 mL, 5.0 mmol) in dry dimethylformamide (10 mL), and the reaction mixture was stirred at room temperature for 30 min under argon. The reaction mixture was cooled in an ice bath and a suspension of 107 (˜1.25 g, ˜7.5 mmol) and diisopropylethylamine (1.76 mL, 10.1 mmol) in dry dimethylformamide (10 mL) was added slowly dropwise to the reaction vessel, then the reaction mixture was warmed to room temperature and stirred for 30 min under argon. The reaction mixture was purified by preparative HPLC in 10-100% acetonitrile/0.1% formic acid to yield 108 (0.56 g, 16%, ESI m/z=679.50 [M+H]⁺).

### l. Preparation of Peptide Resin 109

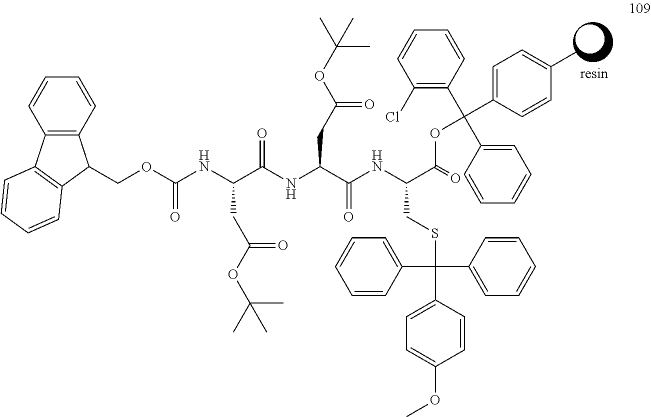 [SMILES: COc1ccc(C(SC[C@H](NC(=O)[C@H](CC(=O)OC(C)(C)C)NC(=O)[C@H](CC(=O)OC(C)(C)C)NC(=O)OCC2c3ccccc3-c3ccccc32)C(=O)OC(c2ccccc2)(c2ccc(C)cc2)c2ccccc2Cl)(c2ccccc2)c2ccccc2)cc1]

TABLE 1
Reagents for peptide 109 synthesis

| | | | Molecular | |
|---|---|---|---|---|
| | | | weight | |
| Reagent | mmol | Equivalents | (g/mol) | quantity |
| H-Cys(4- | 0.87 | 1.0 | | |
| methoxytrityl)-2- | | | | |
| chlorotrityl-Resin | | | | |
| Fmoc-Asp(OtBu)—OH | 2 × 1.74 | 2 × 2.0 | 411.5 | 716 mg |
| PyBOP | 2 × 1.73 | 2 × 2.0 | 520.39 | 900 mg |
| diisopropylethylamine | 2 × 3.48 | 2 × 4.0 | 129.25 | 606 μL |
| | | | (d = 0.742 | |
| | | | g/mL) | |

In a peptide synthesis vessel H-Cys(4-methoxytrityl)-2-chlorotrityl-resin (0.87 mmol) was loaded and washed with isopropyl alcohol (3×10 mL) followed by dimethylformamide (3×10 mL). To the vessel was then introduced Fmoc-Asp(OtBu)-OH (2.0 equiv) in dimethylformamide, diisopropylethylamine (4.0 equiv), and PyBOP (2.0 equiv). Argon was bubbled for 1 hr, the coupling solution was drained, and the resin was washed with dimethylformamide (3×10 mL) and isopropyl alcohol (3×10 mL). Kaiser tests were performed to assess reaction completion. Fmoc deprotection was carried out using 20% piperidine in dimethylformamide (3×10 mL) before each amino acid coupling. The above sequence was repeated to complete 2 coupling steps. The resin was dried under argon for 30 min.

### m. Preparation of Peptide 110

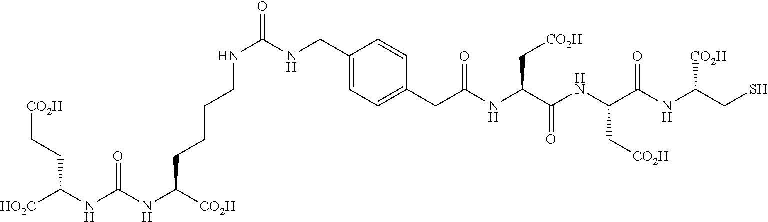 [SMILES: O=C(O)CC[C@H](NC(=O)N[C@@H](CCCCNC(=O)NCc1ccc(CC(=O)N[C@@H](CC(=O)O)C(=O)N[C@@H](CC(=O)O)C(=O)N[C@@H](CS)C(=O)O)cc1)C(=O)O)C(=O)O]

TABLE 2
Reagents for peptide 110 synthesis

| | | | Molecular | |
|---|---|---|---|---|
| Reagent | mmol | Equivalents | weight (g/mol) | quantity |
| Fmoc-Asp(OtBu)- | 0.18 | 1.0 | | |
| Asp(OtBu)-Cys(Mmt)- | | | | |
| 2-ClTrt-resin | | | | |
| 108 | 0.22 | 1.2 | 678.81 | 150 mg |
| PyBOP | 0.37 | 2.0 | 520.39 | 191 mg |
| diisopropylethylamine | 0.74 | 4.0 | 129.25 | 128 μL |
| | | | (d = 0.742 g/mL) | |

In a peptide synthesis vessel 109 (0.18 mmol) was loaded and washed with isopropyl alcohol (3×10 mL) followed by dimethylformamide (3×10 mL). Fmoc deprotection was carried out using 20% piperidine in dimethylformamide (3×10 mL). Kaiser tests were performed to assess reaction completion. To the vessel was then introduced 108 (1.2 equiv) in dimethylformamide, diisopropylethylamine (4.0 equiv), and PyBOP (2.0 equiv). Argon was bubbled for 1 hr, the coupling solution was drained, and the resin was washed with dimethylformamide (3×10 mL) and isopropyl alcohol (3×10 mL). Kaiser tests were performed to assess reaction completion. Peptide was cleaved from the resin using a cleavage mixture consisting of dithiothreitol (114 mg, 0.74 mmol) dissolved in a solution of trifluoroacetic acid (19 mL), H₂O (0.5 mL), triisopropylsilane (0.5 mL). One-third of the cleavage mixture was introduced and argon was bubbled for 30 min. The cleavage mixture was drained into a clean flask. The resin was bubbled 2 more times with more cleavage mixture, for 30 min each, and drained into a clean flask. The drained cleavage mixture was then concentrated and purified by preparative HPLC in 0-30% acetonitrile/0.1% formic acid to yield 110 (66.9 mg, 43%, ESI m/z=844.57 [M+H]⁺).

### n. Preparation of Compound 112 (EC1169)

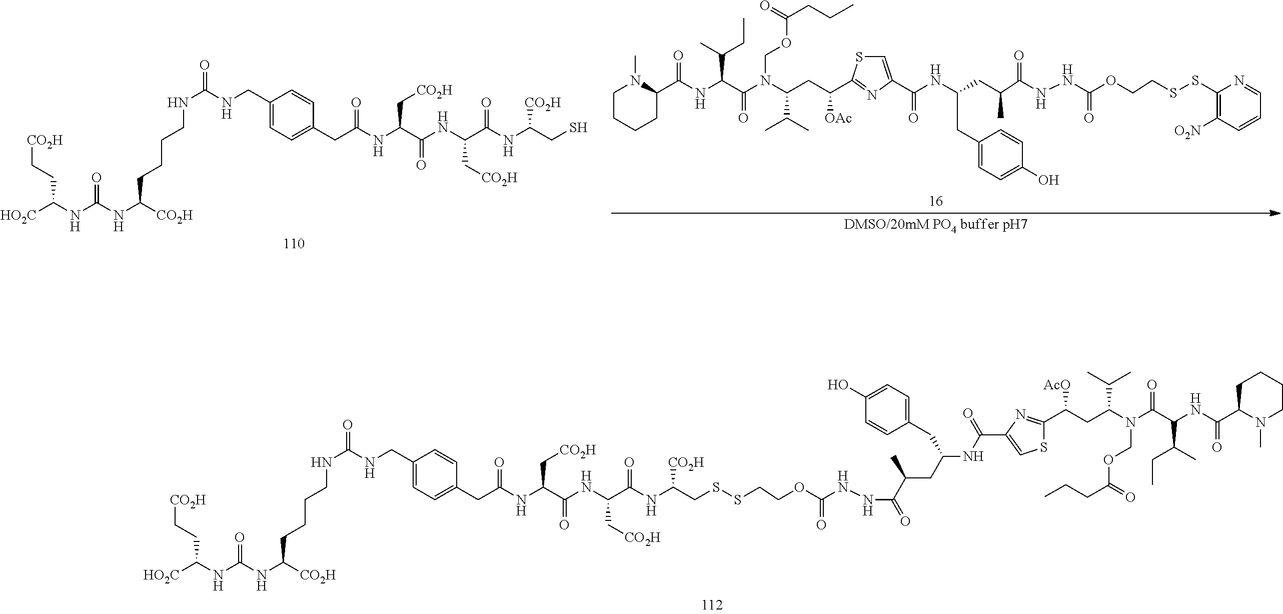

In a 25 mL round bottom flask, 16 (47 mg, 0.04 mmol) was dissolved in dimethylsulfoxide (2 mL). A solution of 110 (36 mg, 0.04 mmol) in 20 mM pH7 sodium phosphate buffer (2 mL) was added dropwise, stirring at room temperature with Argon bubbling for 30 min. The reaction mixture was purified by preparative HPLC (10-100% acetonitrile/50 mM NH₄HCO₃ pH7) to yield compound 112 (56.6 mg, 74%, ESI m/z=895.58 [M+2H]²⁺).

### 2. Preparation of PSMA Imaging Conjugate IIa

PSMA imaging conjugate IIa was prepared according to the following scheme as taught in US patent publication number US20100324008 A1, which is incorporated herein by reference. Specifically, PSMA imaging conjugate IIa was prepared according to the following method.

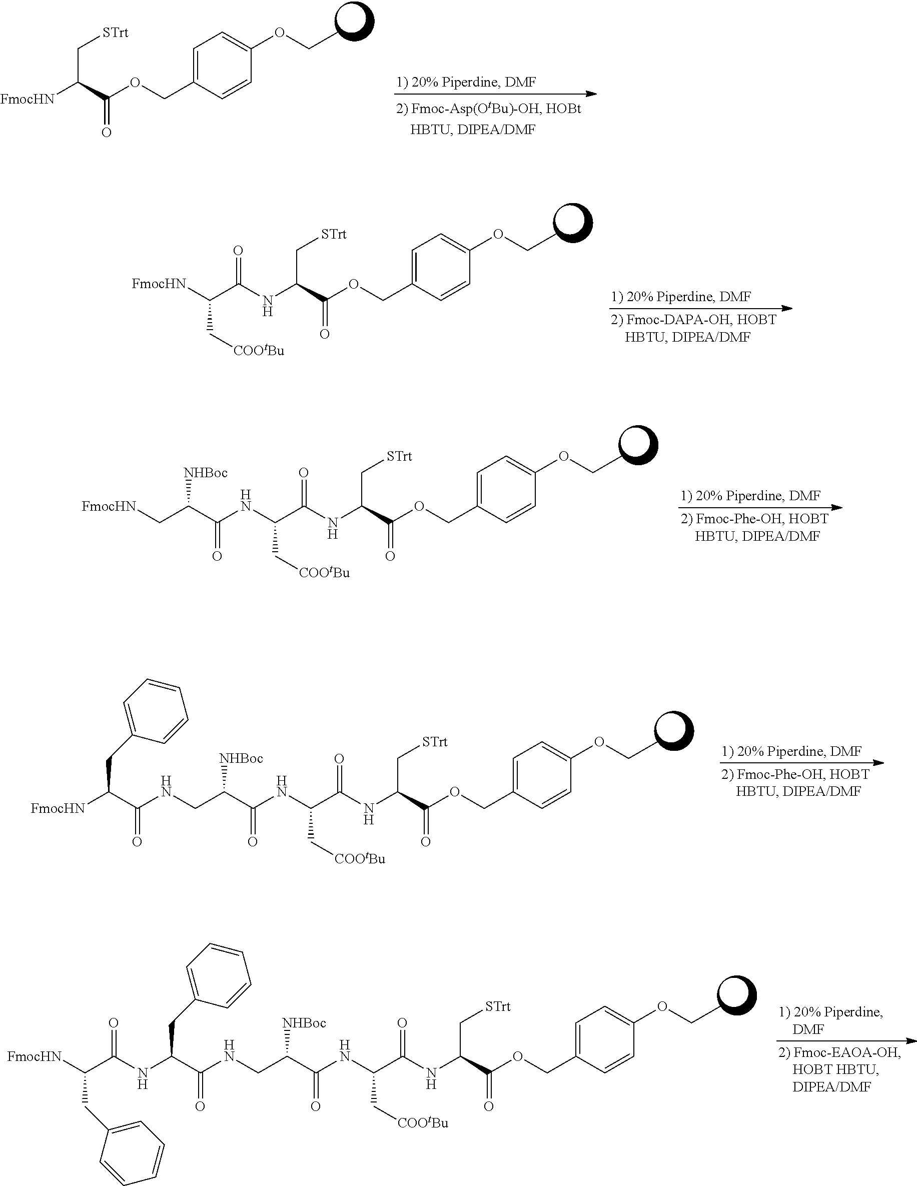

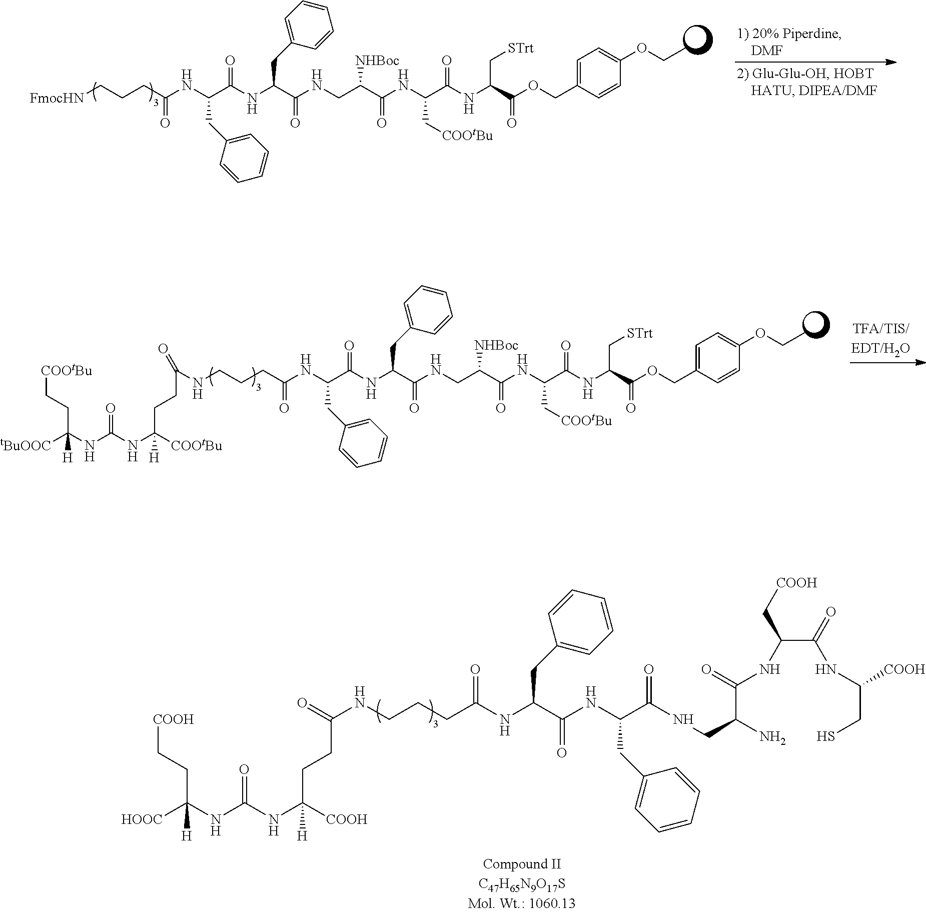

PSMA imaging conjugate IIa was synthesized using standard fluorenylmethyloxycarbonyl (Fmoc) solid phase peptide synthesis (SPPS) starting from Fmoc-Cys(Trt)-Wang resin (Novabiochem; Catalog #04-12-2050). PSMA imaging conjugate II was purified using reverse phase preparative HPLC (Waters, xTerra C₁₈ 10 μm; 19×250 mm) A=0.1 TFA, B=Acetonitrile (ACN); λ=257 nm; Solvent gradient: 5% B to 80% B in 25 min, 80% B wash 30 min run, (61%). Purified compounds were analyzed using reverse phase analytical HPLC (Waters, X-Bridge C₁₈ 5 μm; 3.0×15 mm); A=0.1 TFA, B=ACN; λ=257 nm, 5% B to 80% B in 10 min, 80% B wash 15 min run. C₄₇H₆₅N₂O_{17S}; MW=1060.13 g/mol; white solid; Rt=7.7 min; ¹H NMR (DMSO-d₆/D₂O) δ 0.93 (m, 2H); 1.08 (m, 5H); 1.27 (m, 5H); 1.69 (m, 2H); 1.90 (m, 2H); 1.94 (m, 2H); 2.10 (m, 2H); 2.24 (q, 2H); 2.62 (m, 2H); 2.78 (m, 4H); 2.88 (dd, 1H); 2.96 (t, 2H); 3.01 (dd, 1H); 3.31 (dd, 1H); 3.62 (dd, 1H); 3.80 (q, 1H, aH); 4.07 (m, 1H, aH); 4.37 (m, 1H, aH); 4.42 (m, 2H, aH); 4.66 (m, 1H, aH); 7.18 (m, 10H, Ar—H): LC-MS=1061 (M+H)₊; ESI-MS=1061 (M+H)⁺.

### 3. Preparation of PSMA Imaging Conjugate IIIa

### a. Preparation of PSMA Imaging Conjugate IIa Formulation

A 12 liter volume of Water For Injection (WFI) was sparged with nitrogen. Solutions of 1.0 M NaOH and 0.2 M HCl were prepared and sparged with nitrogen for pH adjustment of the formulation and for preparation of the stannous chloride stock solution. 2000 mL of deoxygenated WFI was added to a 5 L jacketed formulation vessel which was connected to a chiller. The chiller solution was set at 5° C. and circulation was maintained throughout the compounding and filtration process. 88.6 g of sodium gluconate and 1063 mg of EDTA disodium dihydrate were weighed and transferred to the formulation vessel and dissolved. A stannous chloride stock solution at a concentration of 10 mg/mL was made using the previously prepared 0.2 M HCl. A 35.4 mL aliquot of the stannous chloride stock solution was added to the formulation vessel and mixed well with stirring. 354.3 mg (net content) of Compound II was weighed and transferred into the formulation vessel. The mixture was stirred for at least 5 minutes and complete dissolution was observed. The pH was adjusted to 6.8±0.2 with deoxygenated 1.0 M NaOH solution and 0.2 N HCl solution. Deoxygenated WFI was then added until a formulation weight of 3578 g (3543 mL) was achieved. The formulation solution was stirred for five minutes and then sterile filtered through a 0.22 μm filter into a receiving vessel. Vials were filled with 1.01 g±0.03 g (1.00 mL) solution per vial. The vials were loaded into the lyophilizer. Inert atmosphere via a nitrogen blanket was maintained throughout formulation and vialing. Upon completion of the lyophilization cycle, vials were backfilled with nitrogen to approximately 646,000 mTorr. The vials were stoppered and removed from the lyophilizer, crimped with aluminum seals and labeled. Vials were placed in boxes and were stored at 5±3° C.

### b. Room Temperature Labeling of Compound II with ^{99m}Tc to Provide Preparation of PSMA Imaging Conjugate IIIa

A PSMA imaging conjugate IIa kit vial was removed from the refrigerator and allowed to warm to room temperature (17-27° C.) for 15-30 min. The vial was put into a suitable radioactive shielding container. One to Two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then allowed to stand at ambient temperature (17-27° C.) for 15 minutes. 5-6 mL of 0.9% sodium chloride injection, USP, was then added to the vial. The labeled solution was stored at room temperature (17-27° C.) and used within 6 hours of preparation.

### 4. Clinical Biological Examples

### a. Study Design

This is a Phase 1, multicenter, open-label, non-randomized, dose-escalation oncology study in which EC1169 was dosed three times weekly (TIW), on Weeks 1 and 2 of a 3-week schedule or once weekly dosing (QW) on Weeks 1 and 2 of a 3-week schedule to assess toxicity, safety and preliminary efficacy results in patients with metastatic, castration-resistant prostate cancer (mCRPC) who have progressed on abiraterone and/or enzalutamide, and have been previously treated with a taxane except in cases of contraindication (e.g. poor performance status, age or personal choice). Patients were also dosed with 99mTc-EC0652 and underwent a SPECT/CT scan (or SPECT scan if no SPECT/CT is available) to evaluate EC0652 as an imaging agent to identify PSMA expression.

Prior dose escalation methodology for both schedules was based the continuous reassessment method (CRM), in which 1 patient is assigned to 1 dose level. Subsequent patients were to be enrolled upon the first observation of DLT, at which time enrollment to that dose level would be expanded to a maximum of 6 patients. An alternative dose escalation methodology for both schedules was based upon the standard “3+3” approach, in which a minimum of 3 patients is enrolled to a given dose level. Following the first observation of a DLT, the dose level is then expanded to a maximum of 6 patients.

### b. Study Population

### I. Inclusion Criteria

To qualify for enrollment, the following criteria must be met:

1. Patients must have the ability to understand and sign an approved informed consent form (ICF).
2. Patients must be ≧18 years of age.
3. Patients must have histological, pathological, and/or cytological confirmation of prostate cancer.
4. Patients must have progressive, metastatic, castration-resistant prostate cancer (mCRPC) as defined below:

Documented progressive metastatic CRPC will be based on at least one of the following criteria:

a. PSA progression defined as 25% increase over baseline value with an increase in the absolute value of at least 2 ng/mL that is confirmed by another PSA level with a minimum of a 1 week interval and a minimum PSA of 2 ng/mL.

b. Soft-tissue progression defined as an increase ≧20% in the sum of the longest diameter (LD) of all target lesions based on the smallest sum LD since treatment started or the appearance of one or more new lesions.

c. Progression of bone disease (evaluable disease) or (new bone lesion(s)) by bone scan.

5. Patients must have prior and/or ongoing androgen-deprivation therapy and a castrate level of serum testosterone (<50 ng/dL).
6. Patients must have progressed on abiraterone and/or enzalutamide.
7. Patients must have been previously treated with a taxane except in cases of contraindication (e.g. poor performance status, age or personal choice).
8. Patients must have an Eastern Cooperative Oncology Group (ECOG) performance status of 0 or 1.
9. Patients must have at least one lesion that can be followed for disease response assessment on baseline imaging obtained no more than 28 days prior to beginning study therapy. Baseline and follow up radiological disease assessments must include bone scans performed with either Technetium-99m labeled diphosphonates or Fluorine-18 sodium fluoride PET or PET/CT, as per the local standard of care for patients with prostate cancer.
10. Patients with CNS metastases that were symptomatic must have received therapy (surgery, XRT, gamma knife) and been neurologically stable and off of steroids. The patient were off steroids at least 14 days before pre-registration. Asymptomatic CNS metastatic disease without associated edema, shift, requirement for steroids or anti-seizure medications are eligible after discussion with the sponsor medical monitor. For patients with a history of CNS metastasis, baseline and subsequent radiological imaging included evaluation of the brain (MRI preferred or CT with contrast).
11. Patients must have recovered (to baseline/stabilization) from prior therapy-associated acute toxicities.
12. Patients with prior radiation therapy were eligible if they met the following criteria:

a) Previous radiation therapy is allowed to <25% of the bone marrow (Cristy and Eckerman, 1987).

b) Prior radiotherapy must have been completed at least 4 weeks before patient began study therapy.

c) Patient must have recovered from the acute toxic effects of the treatment before beginning study therapy.

13. Patients must have had adequate organ function:

a) Bone marrow reserve: Absolute neutrophil count (ANC)≧1.5×109/L. Platelets≧100×10⁹/L. Hemoglobin≧9 g/dL.

b) Cardiac:
i) Left ventricular ejection fraction (LVEF) equal to or greater than the institutional lower limit of normal. LVEF must be evaluated within 28 days prior to beginning study therapy.
ii) Cardiac Troponin I within normal limit.

c) Hepatic: Total bilirubin≦1.5×the upper limit of normal (ULN). Alanine aminotransferase (ALT), aspartate aminotransferase (AST)≦3.0×ULN OR≦5.0×ULN for patients with liver metastases.

d) Renal: Serum creatinine≦1.5×ULN, or for patients with serum creatinine>1.5 ULN, creatinine clearance≧50 mL/min.

### II. Exclusion Criteria

The presence of any of the following will exclude patients from the study:

1. More than 3 prior systemic anti-cancer therapies (e.g., cytotoxic agents, biologic agents) regimens for metastatic disease.
2. Previous treatment with Samarium-153 or Strontium-89.
3. Any systemic anti-cancer therapy (e.g., chemotherapy, immunotherapy or biological therapy [including monoclonal antibodies] within 28 days prior to beginning study therapy.
4. Known hypersensitivity to the components of the study therapy or its analogs.
5. Carcinomatous meningitis and/or symptomatic central nervous system (CNS) metastases.
6. Malignancies that are expected to alter life expectancy or may interfere with disease assessment. Patients with adequately treated non-melanoma skin cancer and patients with prior history of malignancy who have been disease free for more than 3 years are eligible.
7. Neuropathy CTCAE grade>2.
8. QTc interval of >480 ms.
9. History of ischemic cardiac disease that has occurred within six months prior to study entry.
10. Any other serious cardiac illness or medical conditions such as unstable angina, pulmonary embolism, or uncontrolled hypertension.
11. Other concurrent chemotherapy, immunotherapy, radiotherapy, or investigational therapy.
12. Active uncontrolled infections.
13. Known active Hepatitis B or C infections

### Treatments & Regimens:

### I. PSMA Imaging Agent Ma Administration and Imaging:

Patients received an injection of 0.1 mg of PSMA Imaging Agent IIa labeled with 20-25 mCi of technetium-^{99m} (PSMA Imaging Agent Ma). Patients were subjected to SPECT/CT imaging of the region(s) known to contain target lesion(s) approximately 3-4 hours after injection of PSMA Imaging Agent Ma (^{99m}Tc-EC0652). For sites where SPECT/CT imaging is not available, SPECT imaging alone was carried out.

II. PSMA-Tubulysin Compound I Administration: PSMA ligand-tubulysin compound I was administered at least 4 days after PSMA Imaging Agent Ma was administered, as an intravenous bolus injection, TIW on Weeks 1 and 2 (i.e., on Days 1, 3, 5, 8, 10, 12 of a 3-week cycle) or once weekly on Weeks 1 and 2 of a 3-week schedule.

### II. Schedule #1 TIW Dosing Dose Escalation:

The starting dose of EC1169 on Schedule 1 was 0.2 mg/m² The table below outlines the dose levels for PSMA ligand-tubulysin compound I for Schedule #1 TIW dosing, with up to 14 doses levels of PSMA ligand-tubulysin compound I planned. Should the MTD not be determined after escalation of PSMA ligand-tubulysin compound I to dose level 6, PSMA ligand-tubulysin compound I may continue to be dose escalated in 25% increments.

Schedule #1 Dose Escalation Scheme for PSMA Ligand-Tubulysin Compound I

| | | Incremental increase |
|---|---|---|
| Level | Dose (mg/m²) | over prior dose level (%) |
| 1 | 0.2 | — |
| 2 | 0.3 | 50 |
| 3 | 0.45 | 50 |
| 4 | 0.6 | 33 |
| 5 | 0.8 | 33 |
| 6 | 1.0 | 25 |
| 7 | 1.3 | 30 |
| 8 | 1.7 | 30 |
| 9 | 2.2 | 30 |
| 10 | 3.0 | 30 |
| 11 | 4.0 | 30 |
| 12 | 5.0 | 30 |
| 13 | 6.5 | 30 |
| 14 | 8.5 | 30 |

### III. Schedule #2 Once Weekly Dosing Dose Escalation Scheme:

The starting dose of EC1169 was 0.30 mg/m². The table below outlines the dose levels for PSMA ligand-tubulysin compound I for Schedule #2 TIW dosing, with up to 14 doses levels of PSMA ligand-tubulysin compound I planned. Should the MTD not be determined after escalation of PSMA ligand-tubulysin compound I to dose level 6, PSMA ligand-tubulysin compound I may continue to be dose escalated in 25% increments.

Schedule #2 Dose Escalation Scheme for PSMA Ligand-Tubulysin Compound I

| | | Incremental increase |
|---|---|---|
| Level | Dosage (mg/m2) | over prior dose level (%) |
| 1 | 0.3 | — |
| 2 | 0.45 | 50 |
| 3 | 0.6 | 50 |
| 4 | 0.8 | 33 |
| 5 | 1.0 | 25 |
| 6 | 1.25 | 25 |
| 7 | 2.0 | 60 |
| 8 | 2.8 | 40 |
| 9 | 3.8 | 33 |
| 10 | 5.0 | 30 |
| 11 | 6.5 | 30 |
| 12 | 8.5 | 30 |
| 13 | 11.0 | 30 |
| 14 | 14.0 | 30 |

### IVA: DLT Definition for Both Schedules:

DLTs will be based on events occurring in Part A during the first cycle of therapy and the adverse events must be drug related (i.e. definitely, probably or possibly):
≧Grade 4 hematological toxicity.
Grade 3 neutropenia with fever>38.5° C. and/or infection requiring antibiotic or antifungal treatment.
Grade 3 neutropenia persisting for >5 days.
Grade 3 thrombocytopenia with clinically significant hemorrhage requiring platelet transfusion.
≧Grade 3 non-hematological toxicity. Grade 3 laboratory abnormalities (e.g., K or Mg) that persist for less than 48 hours are not considered DLTs.
Grade 3 nausea, vomiting lasting more than 72 hours.
A delay of >2 weeks in the scheduled administration of Compound I due to drug-related toxicity.

### IVB: Schedule Specific DLT's:

Schedule #1: TIW Dosing Schedule: Inability to administer at least 4 of the 6 scheduled doses of PSMA ligand-tubulysin compound I in a cycle due to drug-related toxicity.
Schedule #2: Once Weekly Dosing Schedule: Inability to administer both scheduled doses of PSMA ligand-tubulysin compound I in a cycle due to drug-related toxicity.

In the event that a patient drops out resulting in the lack of information about the DLT within the required observation period, the patient will be replaced by a new one, who will be enrolled at the exact same dose.

### V. Route of Administration:

PSMA imaging conjugate IIIa and PSMA ligand-tubulysin compound I was administered via IV bolus injection.

### Results:

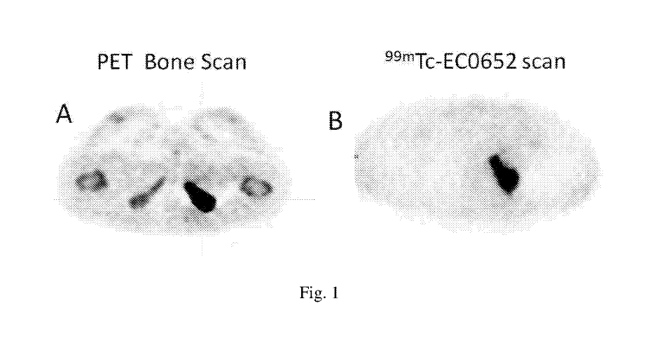

PSMA-imaging conjugate IIIa (^{99m}Tc-EC0652) showed good uptake in tumor lesions (See FIGS. 1A and 1B).

Twenty-one patients were evaluated for cycle 1 toxicity (Schedule #1: 5 pts; Schedule 42: 16 pts). All patients were Caucasian. The median age was 69.0 (range: 53-82). The median time on the study was 6.7 weeks (range: 0.1-33.1). The median number (range) of administered EC1169 cycles was 4 (3-4) for Schedule #1 patients, and 2 (1-12) for Schedule #2 patients. 9 of 21 pts had drug-related (DR) adverse events (AE's). No DRAE occurred in >1 Schedule #1 patient, Vomiting and fatigue were the only DRAE's reported in ≧2 Schedule #2 patients. There were no on-study deaths, DR Grade 3-4 toxicity or serious adverse events, or occurrences of dose limiting toxicity. 3 patients demonstrated stable disease lasting more than 4 cycles. No patient demonstrated a 50% decrease in PSA. Dose proportionate increases in both Cmax and AUC for EC1169 were observed. Both EC1169 and ^{99m}Tc-EC0652 were well tolerated in mCRPC patients.

1. A method for treating a cancer in a patient in need of such treatment comprising, administering to the patient a therapeutically effective amount of a PSMA ligand-tubulysin compound I 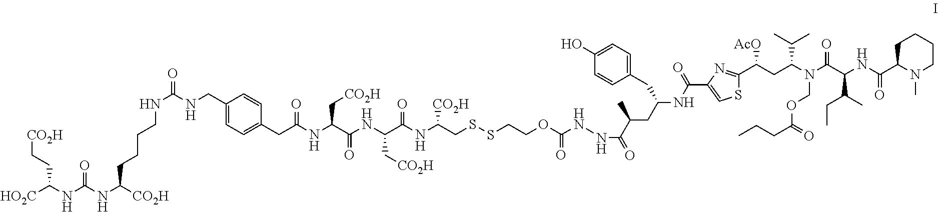 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CC(=O)O)NC(=O)[C@H](CC(=O)O)NC(=O)Cc2ccc(CNC(=O)NCCCC[C@H](NC(=O)N[C@@H](CCC(=O)O)C(=O)O)C(=O)O)cc2)C(=O)O)cs1)C(C)C]
or a pharmaceutically acceptable salt thereof.

2.-5. (canceled)

6. The method of claim 1, wherein the cancer is a primary brain cancer, a secondary brain cancer, metastatic prostate cancer.

7.-17. (canceled)

18. The method of claim 1, further comprising imaging PSMA expression by the cancer.

19. The method of claim 18, wherein the imaging occurs before the step of administering.

20. The method of claim 19, wherein the imaging is performed by imaging and wherein the imaging is selected from the group consisting of SPECT imaging, PET imaging, IHC, and FISH.

21. The method of claim 20, wherein the imaging is performed by SPECT imaging.

22. The method of claim 1, further comprising determining the PSMA status of the patient by imaging.

23. The method of claim 22, wherein the imaging is SPECT imaging.

24. The method of claim 23, wherein the PSMA status of the patient correlates with a clinical benefit to the patient.

25. The method of claim 24, wherein the clinical benefit is selected from the group consisting of inhibition of tumor growth, stable disease, a partial response, and a complete response.

26. The method of claim 25, wherein the clinical benefit is stable disease.

27. The method of claim 24, wherein at least one PSMA positive lesion indicates functionally active PSMA.

28. The method of claim 18, wherein the imaging comprises administering to the patient a PSMA ligand-imaging conjugate of the formula II 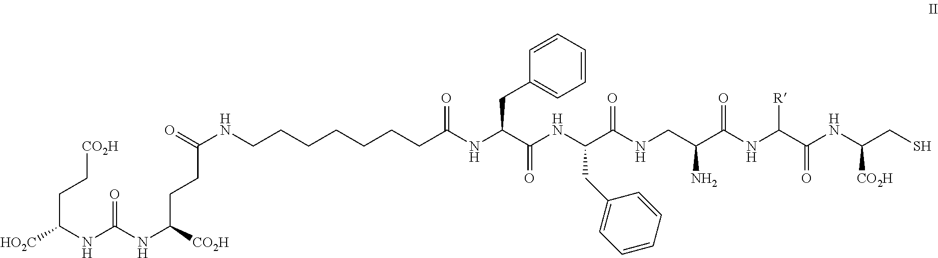
or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

29.-31. (canceled)

32. The method of claim 28, wherein the PSMA ligand-imaging conjugate is of the formula IIa 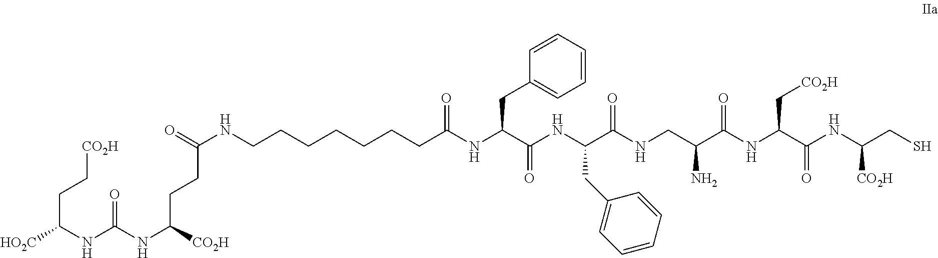 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]
or a pharmaceutically acceptable salt thereof, and wherein a radionuclide is bound to the conjugate.

33. The method of claim 32, wherein the PSMA ligand-imaging conjugate is of the formula IIIa 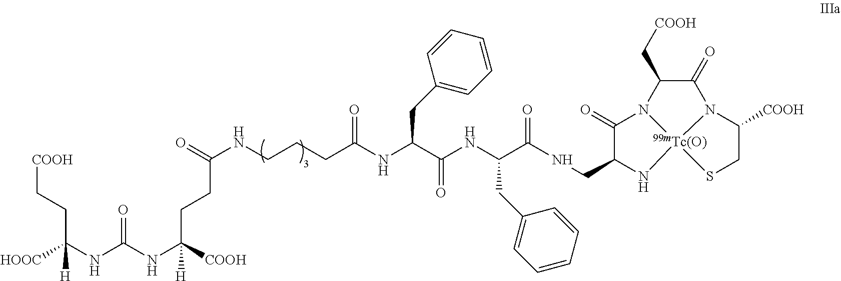
or a pharmaceutically acceptable salt thereof.

34. The method of claim 22, wherein the determining comprises administering to the patient a PSMA ligand-imaging conjugate of the formula II 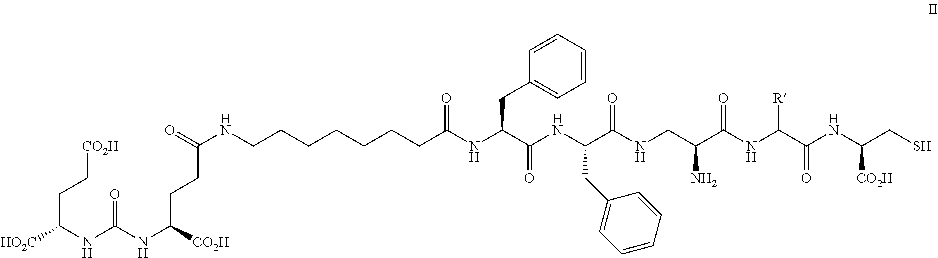
or a pharmaceutically acceptable salt thereof, wherein R′ is hydrogen, or R′ is selected from the group consisting of alkyl, aminoalkyl, carboxyalkyl, hydroxyalkyl, heteroalkyl, aryl, arylalkyl and heteroarylalkyl, each of which is optionally substituted, and wherein a radionuclide is bound to the conjugate.

35.-37. (canceled)

38. The method of claim 34, wherein the PSMA ligand-imaging conjugate is of the formula IIa 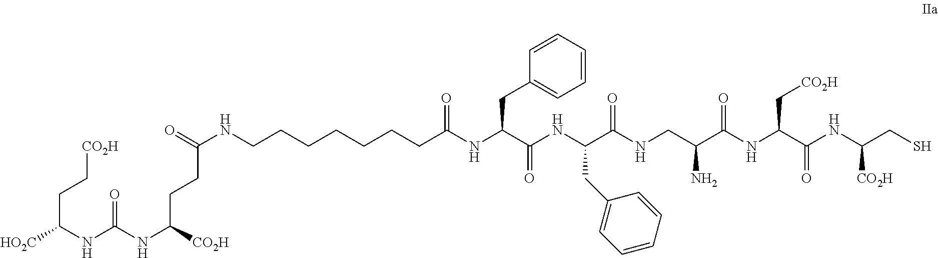 [SMILES: N[C@@H](CNC(=O)[C@H](Cc1ccccc1)NC(=O)[C@H](Cc1ccccc1)NC(=O)CCCCCCCNC(=O)CC[C@H](NC(=O)N[C@@H](CCC(=O)O)C(=O)O)C(=O)O)C(=O)N[C@@H](CC(=O)O)C(=O)N[C@@H](CS)C(=O)O]
or a pharmaceutically acceptable salt thereof, and wherein a radionuclide is bound to the conjugate.

39. The method of claim 38, wherein the PSMA ligand-imaging conjugate is of the formula IIIa 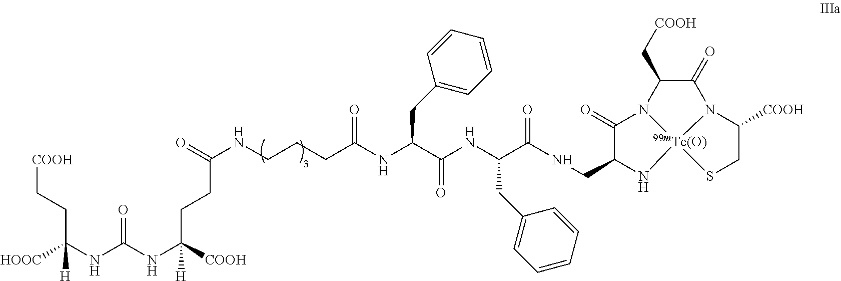
or a pharmaceutically acceptable salt thereof.

40. (canceled)

41. The method of claim 1, wherein the patient has been treated with at least one prior treatment.

42. The method of claim 41, wherein the at least one prior treatment is selected from the group consisting of a chemotherapeutic agent, surgery, radiation therapy, immunotherapy, photodynamic therapy, stem cell therapy, and hyperthermia.

43. (canceled)

44. The method of claim 41, wherein the prior treatment is selected from the group consisting of palifosfamide, 5-fluorouracil, capecitabine, pemetrexed, cisplatin, carboplatin, gemcitabine, paclitaxel, vinorelbine, eribulin, docetaxel, cyclophosphamide, doxorubicin, regorafinib, and combinations thereof.

45.-82. (canceled)
